(19) United States
(12) Patent Application Publication
(10) Pub. No.: US 2026/0345211 A1
(43) Pub. Date: Nov. 5, 2026

(54) BONE ANCHORING, BONE REPLACEMENT, AND BONE SPACING SURGICAL SYSTEMS AND METHODS

(71) Applicant: ANCHORMESH LTD, Rehovot (IL)

(72) Inventors: Liraz SHLOMOFF, Rishon LeZion (IL); Tamir BEN DAVID, Tel-Aviv (IL); Tovy SIVAN, Kfar Saba (IL); Boaz HARARI, Ganei Tikva (IL); David SALTON, Hod-Hasharon (IL)

(21) Appl. No.: 19/718,582

(22) Filed: Jun. 24, 2026

Related U.S. Application Data
(63) Continuation-in-part of application No. PCT/IL2024/051228, filed on Dec. 28, 2024.
(60) Provisional application No. 63/615,685, filed on Dec. 28, 2023.
(60) Provisional application No. 63/837,300, filed on Jul. 2, 2025.

(51) Int. Cl. A61B 17/86

(52) U.S. Cl. CPC A61B 17/8625; A61B 17/8635; A61B 2017/8655

(57) ABSTRACT

A bone anchoring surgical system comprising a bone anchoring device comprising a bone anchor body configured to anchor a bone; and a deformable structure associated with the bone anchor body and located distally or proximally to the bone anchor body, the deformable structure configured to expand following deployment thereof in a target inner body skeleton position.

### CROSS REFERENCE TO RELATED APPLICATIONS

This application is a Continuation-in-Part of PCT Patent Application No. PCT/IL2024/051228 having International filing date of Dec. 28, 2024, which claims the benefit of priority of U.S. Provisional Patent Application No. 63/615,685, filed Dec. 28, 2023. This application also claims the benefit of priority of U.S. Provisional Patent Application No. 63/837,300, filed Jul. 2, 2025. The contents of the aforementioned applications are incorporated herein by reference in their entirety.

### FIELD OF THE INVENTION

The present invention is in the field of medical surgery, in particular bone surgery devices and methods.

### BACKGROUND OF THE INVENTION

Bone anchoring or fixation devices, such as surgical screws are essential components in many orthopedic procedures. Repair of bone fractures and injured ligaments, bone replacements, bone spacers implantations, and joint replacements typically require anchoring elements. Specifically, in some medical conditions there is a benefit to insert a bone connecting element; a bone spacer; and/or a bone replacement element. Such procedures require bone anchoring of the implanted elements, or the repaired tissues. Such connecting and spacing elements can help maintain the normal bone and joint configuration and function by retaining the bone's integrity, normal function and provide support to nearby bones and soft tissues.

The stability of bones during movement is maintained by having natural inter-bone spacers, often with cartilage that provides strength and can absorb and distribute external loads. In some cases, there is a need to replace or to support the natural spacers, in order to maintain normal function.

Osteoarthritis is a disease of the joints such as of the knees; hips; and other joints. For example, osteoarthritis at the base of the thumb (or trapeziometacarpal joint) may cause pain, stiffness, and weakness in the thumb. There are many types of surgeries for the base of the thumb with the goal of reducing pain and increasing function. Arthroplasty techniques have ranged from simple, partial or complete trapezectomy to various implant and ligament interposition and reconstructions. These techniques have been generally indicated for stage II or greater disease once the patient has failed conservative management.

In some trauma or degenerative conditions, there is a need to fuse two or more bony structures together, or to repair bone to muscle and/or ligament interfaces and provide bone tension bands allowing two bone segments' micromovements.

Rotator cuff tears are among the most common shoulder injuries, particularly in adults over 40. Rotator cuff tears involve damage to the group of four muscles and tendons that stabilize the shoulder joint and allow arm movement. Tears can occur from acute injuries, repetitive overhead activities, or age-related wear. Symptoms often include shoulder pain, weakness, and limited range of motion.

Repair of rotator cuff tears is commonly performed using minimally invasive arthroscopic surgery. The procedure involves reattaching the torn tendon to the bone using small anchors and sutures. Post-surgery, physical therapy is essential to regain strength and mobility. Arthroscopic repair offers smaller incisions, less pain, and quicker recovery compared to open surgery.

Currently fixation techniques typically include the use of screws, plates, pins, wires etc. Such fixation techniques can prevent unwanted movement; attach bony structures; mitigate pain; maintain bone integrity; and restore regular function including natural movement and range of motion.

There is a continuous need in bone anchoring devices that will provide an enhanced and more stable fixation of external bone implants and fixation means. There is further a need in advanced bone replacement and bone spacers with improved bone anchoring means. The present invention affords such improved bone surgery and anchoring devices. The present invention further allows improved anchoring of hardware to the bone to prevent cases of pull out of the fixation elements, especially in cases of impaired bony structure and trauma.

### SUMMARY OF THE INVENTION

The present invention relates to bone anchoring devices and systems that may be useful to strengthen and stabilize anchoring elements within bones, for example to strengthen and stabilize screws used to repair bone fractures, or screws that anchor surgical wires in the treatment of bone ligaments. Some of the herein bone anchoring devices and systems may be implemented as bone spacers or bone replacement elements and thereby used in bone prostheses treatments and joint replacements.

According to one aspect of the present invention there is provided a bone anchoring system comprising
a bone anchoring device comprising:
   a bone anchor body configured to anchor a bone; and
   a deformable structure associated with the bone anchor body and located distally or proximally to the bone anchor body; the deformable structure configured to at least laterally expand following deployment thereof in a target inner body skeleton position.

In one or more embodiments, the deformable structure is an expandable structure. In one or more embodiments, the expandable structure is configured to contact the inner soft bone tissue and encourage growth of said tissue into the expandable structure, and by that improve the anchoring forces of the anchoring device.

In one or more embodiments, the expandable structure is a mesh configured to expand following deployment thereof in the inner body position.

In one or more embodiments, the expandable structure is configured to expand by filling thereof with a filler material.

In one or more embodiments, the expandable structure is configured to expand following pushing or pulling forces applied thereon.

In one or more embodiments, the expandable structure is configured to expand when pushed on by the bone anchor body or a portion thereof, and/or a delivery tool of the bone anchoring device.

In one or more embodiments, the expandable structure is self-expanded.

In one or more embodiments, the expandable structure is configured to laterally bulge following deployment thereof in the target inner body position.

In one or more embodiments, the expandable structure is made from a shape memory material. In one or more embodiments, the expandable structure is made from one or more metals. In one or more embodiments, the metal is titanium. In one or more embodiments, the metal is nickel. In one or more embodiments, the expandable structure is made from an alloy.

In one or more embodiments, the shape memory material is selected from the group consisting of shape memory alloys and shape memory polymers.

In one or more embodiments, the shape memory alloy is nitinol.

In one or more embodiments, the system further comprising a delivery tool for delivering the bone anchoring device into the target inner body position.

In one or more embodiments, the delivery tool is configured to position the bone anchor body in the bone, and/or to expand the expandable structure in the target inner body position.

In one or more embodiments, the delivery tool includes one or more of: a rotation mechanism for rotating, or deploying the expandable structure and/or the bone anchor body. In one or more embodiments, the delivery tool includes one of a rotation mechanism for rotating, and/or deploying the bone anchoring device. In one or more embodiments, the delivery tool includes two rotation mechanisms for rotating, and/or deploying the bone anchoring device. In one or more embodiments, the delivery tool includes three rotation mechanisms for rotating, and/or deploying the bone anchoring device. In one or more embodiments, the delivery tool consists of one rotation mechanism for rotating, and/or deploying the bone anchoring device. In one or more embodiments, the delivery tool consists of two rotation mechanisms for rotating, and/or deploying the bone anchoring device. In one or more embodiments, the delivery tool consists of three rotation mechanisms for rotating, and/or deploying the bone anchoring device.

In one or more embodiments, the delivery tool includes a sheath configured to accommodate the expandable structure in a non-expanded state.

In one or more embodiments, upon retraction of the sheath the expandable structure transforms to an expanded state.

In one or more embodiments, the sheath is configured to allow deployment and/or retraction of the expandable structure.

In one or more embodiments, the delivery tool includes an internal delivery tool shaft configured to allow swiveling and/or knocking, and/or delivery of the bone anchoring device into the bone.

In one or more embodiments, the delivery tool includes an injection tube for injecting a filler material into the expandable structure.

In one or more embodiments, the filler material is selected from the group consisting of: bone cement; autologous bone; a bone substitute; a polymer; a ligament; metal particles, a platelet rich plasma (PRO), a bone marrow aspirate concentrate (BMAC), a bone morphogenetic protein (BMP), and a combination thereof.

In one or more embodiments, the expandable structure is disposed distally of the bone anchor body.

In one or more embodiments, the expandable structure is disposed proximally of the bone anchor body.

In one or more embodiments, the system further comprises a suture wire coupler at the distal or proximal end of the bone anchoring device configured to allow insertion therethrough of surgical wires. In one or more embodiments, the suture wire coupler comprises an eyelet/hole.

In one or more embodiments, the bone anchoring device comprises a distal extension with an eyelet/hole, a proximal bone anchor body and an expandable structure disposed therebetween.

In one or more embodiments, the bone anchor body has a screw-like structure.

In one or more embodiments, the screw-like structure is used to expand the expandable structure during screwing thereof into the target inner body position.

In one or more embodiments, the expandable structure is attached to an elongated bar, or wherein the expandable structure includes a cylindrical extension.

In one or more embodiments, the elongated bar or cylindrical extension is configured to connect two parts of a fractured bone.

In one or more embodiments the bone anchoring device comprises a distal bone anchor body, a proximal elongated bar or cylindrical extension and an expandable structure disposed therebetween.

In one or more embodiments, the proximal portion of the elongated bar or cylindrical extension is configured to allow fixation thereof to a bone via one or more bone fixation members.

In one or more embodiments, the one or more bone fixation members comprises a bone anchor screw. In one or more embodiments, the expandable structure is configured to act as a bone-replacement component.

In one or more embodiments, the bone anchoring device comprises a distal bone anchor body and a proximal expandable structure.

In one or more embodiments, the bone anchor body has a hollow cylindrical shape, and the expandable structure is configured to pass through the hollow bone anchor body when the expandable structure is in a non-expanded state and to expand from the bone anchor body when the expandable structure is in an expanded state.

In one or more embodiments, the bone anchor body is configured as a hollow spine screw, and the expandable structure is disposed distally to the hollow spine screw following deployment thereof.

In one or more embodiments, the expandable structure is expanded during screwing of the hollow spine screw.

In one or more embodiments, the hollow bone anchor body is a hollowed spine screw and wherein the expandable structure is configured to pass through the hollowed spine screw from a proximal position to a distal position.

In one or more embodiments, the expandable structure is configured to act as a bone-spacer component comprising a first distal bone anchor body and a second proximal bone anchor body, wherein the expandable structure is disposed between the first and second bone anchor bodies.

In one or more embodiments, the target inner body skeleton position is selected from a bone, a void space of a missing or defected bone or a portion thereof, and a space between bone portions.

In a further aspect, the invention provides a method of performing a bone surgery comprising:
drilling into a bone to form a lumen that is dimensioned to receive at least a portion of a bone anchoring device;
inserting at least a bone anchor body of the bone anchoring device into the lumen of the bone; and
deploying an expandable structure of the anchoring device to thereby expand the expandable structure in a target inner body skeleton position.

In one or more embodiments, the method comprises expanding the expandable structure to bulge laterally.

In one or more embodiments, the target inner body skeleton position is the bone lumen, and the method comprises expanding the expandable structure against the walls of the lumen.

In one or more embodiments, the target inner body skeleton position is a void space of a missing bone, and the method comprises expanding the expandable structure to fill the void space of a missing bone.

In one or more embodiments, the target inner body skeleton position is a space between neighboring bones and the method comprises expanding the expandable structure within the space.

In one or more embodiments, inserting the bone anchor body into the lumen of the bone presses on the expandable structure and causes the expandable structure to expand laterally.

In one or more embodiments, inserting the bone anchor body comprises screwing the bone anchor body into the bone lumen.

In one or more embodiments, inserting the bone anchor body into the lumen comprises using a striking or a knocking action.

In one or more embodiments, inserting the bone anchor body comprises swiveling the bone anchor body into the bone lumen.

In one or more embodiments, the lumen is in the form of a tunnel and after inserting the bone anchor body into the tunnel, the method comprises inserting a connector bar into the tunnel; and screwing one or more auxiliary anchor screws into the wall of the tunnel.

In one or more embodiments, inserting the bone anchor body comprises using a delivery tool.

In one or more embodiments, the method comprises retracting the delivery tool using a sheath initially disposed about the delivery tool.

In one or more embodiments, retracting the sheath causes the expandable structure to self-expand and bulge laterally in the target inner body skeleton position.

In one or more embodiments, the method comprises introducing a filler material into the expandable structure.

In one or more embodiments, the filler material is selected from the group consisting of a bone cement, an autologous bone, a bone substitute, a polymer, and/or a ligament.

In one or more embodiments, the expandable structure comprises a mesh configured to expand following deployment thereof in the inner body skeleton position.

In an aspect of the invention, the invention provides a bone anchoring surgical system comprising
a bone anchoring device configured to anchor a bone, comprising
   a proximal bone anchor body;
   a distal tip; and
   a deformable structure disposed between the proximal bone anchor body and the distal tip;
wherein the deformable structure comprises a plurality of deformable wings configured to laterally expand following their deployment in a target inner body skeleton position.

In one or more embodiments, the bone anchoring device comprises a proximal bone anchor body, a distal tip, and a deformable structure disposed therebetween the bone anchor body and the distal tip.

In one or more embodiments, the deformable structure comprises a first and a second tubular bodies, which overlap with each other and wherein each of the tubular bodies comprises a plurality of deformable wings positioned between two base elements.

In one or more embodiments, the outer diameter of the bone anchor body is larger than the outer diameter of the distal tip of the bone anchoring device, ensuring that the device remains securely anchored within the bone lumen and cannot exit the lumen.

In one or more embodiments, the tip is closed at the distal extremity thereof.

In one or more embodiments, the tip is open at the distal extremity thereof.

In one or more embodiments, the deformable structure comprises an expandible mesh. In one or more embodiments, the deformable structure does not comprise an expandible mesh.

In one or more embodiments, the deformable structure comprises a plurality of deformable wings disposed between a plurality of spaces.

In one or more embodiments, the deformable structure comprises an expandable mesh that encloses the deformable structure, the deformable structure comprises a plurality of expandable wings, optionally disposed between a plurality of longitudinal spaces, allowing the expansion of the mesh.

In one or more embodiments, the bone anchoring device is configured to move between a non-expanded configuration and an expanded configuration. In one or more embodiments. the bone anchoring device is configured to move between a non-expanded configuration and an expanded configuration, wherein in the non-expanded configuration, the wings are held flat in the deformable structure and wherein in the expanded configuration, the wings are extended out of the deformable structure and bulge radially out of the periphery of the bone anchoring device.

In one or more embodiments, wherein in a non-expanded configuration of the bone anchoring device, the wings are held flat in the spaces of the deformable structure and wherein in the expanded configuration of the bone anchoring device, the wings are extended out of the spaces of the deformable structure and the mesh is supported by and radially surround the deformable structure and bulge radially out of the periphery of the bone anchoring device.

In one or more embodiments, the deformable structure is configured to expand and optionally to further reversibly collapse.

In or more embodiments, wherein in a non-expanded configuration of the bone anchoring device, the expandable wings are held flat in the deformable structure and wherein in the expanded configuration of the anchoring device, the expandable wings bulge radially out of the periphery of the anchoring device.

In one or more embodiments, the distal tip, the deformable structure and the bone anchor body are hollow and configured to allow passing therethrough at least one wire(s), the wire(s) extend from the distal tip through the deformable structure and through an opened proximal end of the bone anchor body.

In one or more embodiments, the bone anchoring device further comprises at least one suture wire coupler at its distal tip, the suture wire coupler is configured to couple at least one suture wires to the bone anchoring device.

In one or more embodiments, the suture wire coupler is disposed externally within said tip and said suture wire extending from the distal tip along the exterior of the bone anchoring device, and/or wherein said suture wire coupler is disposed internally within said tip and said suture wire extending from the distal tip along the interior of the bone anchoring device.

In one or more embodiments, the at least one suture wires is slidable along the bone anchoring device. In one or more embodiments, the at least one suture wires is fixedly attached thereto.

In one or more embodiments, the suture wire coupler comprises one or more eyelets. In one or more embodiments, one or more eyelets at the distal tip of the bone anchor body, allows passing therethrough, at least one suture wire(s).

In one or more embodiments, the suture wire coupler comprises one or more pins. In one or more embodiments, one or more pins at the distal tip of the bone anchor body, allows coupling and passing therethrough the at least one suture wire(s).

In one or more embodiments, the mesh is configured to expand following deployment thereof in the inner body position.

In one or more embodiments, the mesh is configured to expand following a pulling force applied on the distal tip.

In one or more embodiments, the deformable structure is configured to deform following a pulling force applied on the distal tip to thereby deform the deformable structure to push out the wings so that the wings outwardly protrude from the bone anchoring device.

In one or more embodiments, the deformable structure is configured to deform when a pulling force is applied by a delivery tool of the bone anchoring device.

In one or more embodiments, the applied pulling force is configured to deform the deformable structure to push out the wings so that the wings outwardly protrude from the periphery of the anchoring device.

In one or more embodiments, the wings support and push out the expandable mesh to form a balloon-like mesh extending out of the plane of the bone anchoring device and radially surround the deformable structure.

In one or more embodiments, the deformable structure is hollowed and configured to be filled with a filler material.

In one or more embodiments, the filler material is selected from the group consisting of: bone cement; autologous bone; a bone substitute; a polymer; a ligament; metal particles; a platelet rich plasma (PRO); a bone marrow aspirate concentrate (BMAC); a bone morphogenetic protein (BMP); and a combination thereof.

In one or more embodiments, the deformable structure comprises three or more expandible wings. In one or more embodiments, the deformable structure comprises four or more expandible wings. In one or more embodiments, the deformable structure comprises five or more expandible wings. In one or more embodiments, the deformable structure comprises six or more expandible wings. In one or more embodiments, the deformable structure comprises seven or more expandible wings. In one or more embodiments, the deformable structure comprises eight or more expandible wings. In one or more embodiments, the deformable structure comprises between four and sixteen wings. In one or more embodiments, the deformable structure comprises between four and twelve wings. In one or more embodiments, the deformable structure comprises between four and ten wings. In one or more embodiments, the wings have a strip like structure that extends along a longitudinal line of the bone anchoring device.

In one or more embodiments, the deformable structure comprises a plurality of external wings and a plurality of internal wings. In one or more embodiments, the deformable structure comprises four external wings and four internal wings.

In one or more embodiments, the deformable structure comprises an outer tubular body and an inner tubular body, the tubular bodies overlap, and wherein each of the tubular bodies comprises a plurality of deformable wings and wherein the outer tubular body comprises a plurality of spaces configured to accommodate/receive the wings of the inner tubular body when the deformable structure is in an expanded state.

In one or more embodiments, the deformable structure includes an outer tubular body and an inner tubular body, each comprising a plurality of deformable wings. In one or more embodiments, the wings of the outer tubular body extend outward from an outer position, and the wings of the inner tubular body extend outward from an inner position relative to the wings of the outer tubular body.

In one or more embodiments, the deformable structure comprises at least one stopper that prevents the wings from being overly deformed. In one or more embodiments, the inner tubular body of the deformable structure comprises at least one stopper that prevents the wings from being overly deformed. In one or more embodiments, the stoppers are arranged between the wings. In one or more embodiments, the stoppers are alternately arranged between the wings, such that a stopper is positioned between every two adjacent wings. In one or more embodiments, each stopper comprises two or more opposing ribs. In one or more embodiments, the stopper ribs restrict the contraction of the deformable structure to the point where two opposing ribs contact each other.

In one or more embodiments, the bone anchoring device, deformable structure, bone anchor body, and/or distal tip is made from a material selected from the group consisting of a shape memory material, a titanium, a cobalt chrome, a stainless steel, an alloy, a metal, a bio-absorbable material, a polymer, and a combination thereof. In one or more embodiments, the bone anchoring device, deformable structure, bone anchor body, and/or distal tip is made from a shape memory material. In one or more embodiments, the shape memory material is selected from the group consisting of shape memory alloys, shape memory polymers, and a combination thereof. In one or more embodiments, the shape memory alloy is nitinol. In one or more embodiments, the metal is titanium. In one or more embodiments, the metal is nickel.

In one or more embodiments, the bone anchor body has an outer screw-like structure. In one or more embodiments, the bone anchor body has a smooth outer surface.

In one or more embodiments, the system further comprises a delivery tool for delivering/inserting the bone anchoring device into the target inner body position. In one or more embodiments, the invention provides a bone anchoring device delivery tool.

In one or more embodiments, the delivery tool is configured to position the bone anchor body in the bone, and to expand the deformable structure in the target inner body position.

In one or more embodiments, the delivery tool comprises:
a delivery tool shaft configured to couple and insert the bone anchoring device into a lumen of the inner body skeleton position; and a
a delivery tool handle for maneuvering the delivery tool to deform the deformable structure of the bone anchoring device within the target body skeleton position.

In one or more embodiments, the shaft and the handle are configured to accommodate one or more wires coupled at one end to the bone anchoring device and at a second end to the proximal end of the delivery device.

In one or more embodiments, the shaft is configured for accommodating a slider therein, the slider coupled to a control knob within the handle configured to slide the slider towards the distal tip of the shaft.

In one or more embodiments, the delivery tool is configured to accommodate at least one suture wire, which is coupled to the bone anchoring device, such that the suture wire extends through the delivery tool, and exits at the proximal end of the delivery tool.

In one or more embodiments, the delivery tool further comprises at least one rotation mechanism comprising a knob configured to expand the deformable structure of the bone anchoring device within the inner body skeleton position.

In one or more embodiments, the shaft is configured for accommodating a slider therein, the slider is coupled at the distal extremity thereof to the bone anchoring device and at the proximal extremity thereof to the rotation mechanism, the rotation mechanism is configured to slide the slider towards the proximal end of the delivery tool and controllably expand the deformable structure of the bone anchoring device within the inner body skeleton position.

In one or more embodiments, the delivery tool further comprises one or more rotation mechanisms comprising a knob configured to release the bone anchoring device from the delivery tool.

In one or more embodiments, the delivery tool further comprises an indicator that indicates the status of expansion of the deformable structure.

In one or more embodiments, the delivery tool further includes a port and an injection tube for injecting a filler material into the deformable structure.

In one or more embodiments, the system further comprising a retrieving tool for retrieving the bone anchoring device out of the target body skeleton position.

In one or more embodiments, the retrieving tool is configured to attach to the bone anchoring device in the bone, and to flatten the deformable structure in the target body position.

In one or more embodiments, the retrieving tool comprises:
a retrieving tool shaft configured for insertion into a lumen of the body, the distal tip of the shaft configured to couple to the bone anchoring device; and
a retrieving tool handle for maneuvering the retrieving tool to remove the bone anchoring device from within the target body position.

In one or more embodiments, the retrieving tool comprises: a retrieving tool shaft configured for insertion into a lumen of the body, the distal tip of the shaft configured to couple to an internal thread of the bone anchor body of the bone anchoring device of the herein invention; and a retrieving tool handle for maneuvering the retrieving tool to remove the anchoring device from within the target body position.

In one or more embodiments, the retrieving tool comprises a slider therein, the slider is coupled to a pushing rod and is moveable between a position protruding out of the proximal end of the handle and a position protruding from the distal end of the shaft, the slider is further coupled to a knob configured to slide the slider, wherein the pushing rod is configured to be pushed by the slider to apply a push force on the bone anchoring device to flatten the wings and the deformable structure in the bone anchoring system.

The invention further pertains to a method of performing a bone surgery comprising:
providing a bone anchoring system as disclosed herein;
drilling into a bone to form a lumen that is dimensioned to receive at least a portion of the bone anchoring device;
inserting at least the distal tip of the bone anchoring device into the lumen of the bone; and
deforming the deformable structure of the bone anchoring device to thereby laterally expand the wings of the deformable structure against the walls of the lumen of the bone.

The invention further pertains to a method of treating an orthopedic indication, comprising:
providing a bone anchoring device as disclosed herein;
drilling into a bone to form a lumen that is dimensioned to receive at least a portion of the bone anchoring device;
inserting at least the distal tip of the bone anchoring device into the lumen of the bone; and
deforming the deformable structure of the bone anchoring device to thereby laterally expand the wings of the deformable structure against the walls of the lumen of the bone.

In one or more embodiments, inserting at least the distal tip of the bone anchoring device further comprises screwing, or pushing the bone anchor body of the bone anchoring device into the bone lumen.

In one or more embodiments, inserting the bone anchoring device is utilized using a delivery tool.

In one or more embodiments, the step of deforming the deformable structure of the bone anchoring device is conducted by rotating a rotation mechanism of a delivery tool to thereby push the bone anchoring device proximally to press on the deformable structure and cause the deformable structure to expand laterally.

In one or more embodiments, the method further comprising introducing a filler material into the deformable structure.

In one or more embodiments, the filler material is selected from the group consisting of a bone cement, an autologous bone, a bone substitute, a polymer, and/or a ligament.

Exemplary orthopedic indications of the herein device and method include: repair of tendon or ligament tissue tears (e.g., repair of rotation cuff tears), tendon-to-bone attachment, muscle-to-bone attachment, bone-to-bone, attachment, replacement of the trapezium bone in the hand; connecting bones together, such as a lunate scaphoid connector, and repairing broken bones like the clavicle. The invention is further useful in connecting bones together, repairing broken bones, treating orthopedic oncology indications, treating pediatric orthopedics indications, treating injuries associated with sports, shoulder surgeries, knee surgeries, spine surgeries, hand surgeries, foot surgeries, or hip joint fractures. The invention may also help treat degenerative diseases like osteoarthritis, as well as help in trauma cases and joint replacement.

An aspect of the invention pertains to a delivery tool for inserting and/or deploying within the bone a bone anchoring device, comprising:
a delivery shaft configured to couple to a bone anchoring device;
a delivery handle connected to a proximal end of the delivery shaft; and
a rotation mechanism on the delivery handle configured to expand a deformable structure of the bone anchoring device when actuated.

In one or more embodiments, the delivery tool further comprises a delivery slider disposed within the delivery shaft and operatively connected to the rotation mechanism, wherein the delivery slider is configured to move axially within the delivery shaft when the rotation mechanism is actuated.

In one or more embodiments, the rotation of the rotation mechanism causes axial movement of the delivery slider.

In one or more embodiments, the delivery tool further comprising a rotating member coupling the rotation mechanism to the delivery slider, the rotating member is configured to convert rotational motion of the rotation mechanism into linear motion of the delivery slider.

In one or more embodiments, the delivery tool further comprising a deployment indicator configured to provide visual feedback on the position of the delivery slider.

In one or more embodiments, the delivery shaft comprises a shaft thread configured to engage with a corresponding first thread on a bone anchoring device.

In one or more embodiments, the slider comprises a slider thread configured to engage with a corresponding second thread on a bone anchoring device.

In one or more embodiments, the delivery tool is configured for simultaneous disengagement of both threads from the bone anchoring device.

In one or more embodiments, the shaft thread and the slider thread have similar thread pitches allowing for simultaneous disengagement of both threads from the bone anchoring device.

In one or more embodiments, the rotation mechanism is a rotating knob.

In one or more embodiments, the delivery tool is configured to detach from the bone anchoring device through a swiveling motion, the swiveling motion allows simultaneous disengagement of the shaft thread of the shaft from the first thread of the bone anchoring device and the slider thread on the slider from a second thread of the bone anchoring device.

An aspect of the inventio pertains to a bone anchoring surgical system comprising a bone anchoring device and a delivery too as herein disclosed.

### BRIEF DESCRIPTION OF THE DRAWINGS

The invention may be more clearly understood upon reading of the following detailed description of non-limiting exemplary embodiments thereof, with reference to the following drawings, in which:

FIGS. 1A-1H show schematic side views of bone anchoring surgical systems, in various stages of implementation, in accordance with embodiments of the present invention.

FIGS. 2A-2C show perspective and side views of another embodiment of the present device, which includes a distal eyelet, a mesh, and a bone anchor body with or without a locking element.

FIGS. 3A-3F are perspective views of another embodiment of the present device, useful for rotator cuff/biceps tendon repair and shoulder injuries repair.

FIGS. 4A-4C are perspective views of another embodiment of the present device, useful for bone fracture repair.

FIGS. 5A-5D are perspective views of the device of FIGS. 4A-4C being implemented in a lumen of the inter-medullary canal, with or without reaming, for repairing a clavicle fracture.

FIGS. 6A-6D are perspective views of the device of FIGS. 4A-4C being implemented in a lumen of a bone, for repairing a fibula fracture.

FIG. 7 is a perspective view of another embodiment of the present device, useful for bone replacement.

FIGS. 8A-8L show the device of FIG. 7 implemented in a trapezium replacement procedure.

FIGS. 9A-9C show schematic side views of another embodiment of the present device, in various stages of implementation, useful for spinal (pedicle) surgery.

FIGS. 10A-10B are perspective views of another embodiment of the present device, useful for spinal (pedicle) surgery.

FIGS. 11-11D are perspective views of the device of FIGS. 10A-10B being implemented to repair a spine (pedicle) surgery.

FIG. 12 is a perspective view of an exemplary bone anchoring device implemented as a bone spacer.

FIGS. 13A-13B are perspective views of another exemplary bone anchoring device implemented as a bone spacer.

FIGS. 14A-14B are perspective views of exemplary delivery tools for the present device.

FIG. 15 is a perspective view of another exemplary delivery tool for the present device.

FIGS. 16A-16I illustrate an exemplary bone anchoring device according to an aspect of the present invention.

FIGS. 17A and 17B are perspective and side views of at least part of an exemplary bone anchoring device according to an aspect of the present invention.

FIGS. 18A and 18B are perspective and side views of an exemplary bone anchoring device in a configuration with an expanded mesh according to an aspect of the present invention.

FIGS. 19A and 19B are cross section views of an exemplary bone anchoring device according to an aspect of the present invention.

FIGS. 20A-20E are side and cross section views of an exemplary delivery device according to an aspect of the present invention.

FIGS. 21A-21C are a perspective views of an enlarged proximal portion of an exemplary delivery device according to an aspect of the present invention.

FIGS. 22A-22E are side and perspective views of steps in insertion and an implanted exemplary of bone anchoring device according to an aspect of the present invention.

FIGS. 23A-23D are side, and cross section views of an exemplary retrieving tool according to an aspect of the present invention.

FIGS. 24A and 24B are cross section views of an exemplary retrieving device coupled to an exemplary bone anchoring device according to an aspect of the present invention.

FIGS. 25A-25F are perspective views of steps in the retrieval of an exemplary implanted bone anchoring device according to an aspect of the present invention.

FIGS. 26A-26D are an isometric view of bone anchoring device when in a collapsed/non-expanded state (FIG. 26A); an isometric view of anchoring device when in an expanded state (FIG. 26B); a cross section view of anchoring device in the collapsed/non-expanded state (FIG. 26C); and a cross section view of anchoring device in the expanded state (FIG. 26D).

FIGS. 27A-27C are an isometric view of bone anchoring device when in a collapsed state and coupled to a delivery tool (FIG. 27A); an isometric view of bone anchoring device when in an expanded state and coupled to a delivery tool (FIG. 27B); and an exemplary handle of bone anchoring device delivery tool (FIG. 27C).

FIGS. 28A-28B show an enlargement of a cross section view of the bone anchoring device when in a collapsed state and coupled to a delivery tool (FIG. 28A); and an enlargement of a cross section view of bone anchoring device when in an expanded state and coupled to a delivery tool (FIG. 28B).

FIGS. 29A-29B are a cross section view of the bone anchoring device when in a collapsed state and coupled to a delivery tool (FIG. 29A); and a cross section view of bone anchoring device when in an expanded state and coupled to a delivery tool (FIG. 29B).

FIGS. 30A-30D show a perspective view (FIG. 30A) and a cross-section view (FIG. 30B) of a bone anchoring device in a collapsed state, and perspective views (FIGS. 30C and 30D) of a bone anchoring device in an expanded state.

FIGS. 31A-31D show an exemplary delivery tool and portions thereof used to insert a bone anchoring device into a target inner body skeleton position in accordance with embodiments of the present invention.

FIGS. 32A-32C show a perspective view (FIG. 32A); a magnified perspective cross-section view (FIG. 32B); and an exploded perspective view (FIG. 32C) of a bone anchoring device having two overlapping tubular bodies with deformable structures and wings.

FIG. 33 is a side cut view of a bone anchoring device having two overlapping layers of tubular bodies with deformable structures with wings, and stoppers that restrict the extent of wings deformation.

FIGS. 34A-34B are a perspective view (FIG. 34A) and a cut view (FIG. 34B) of a delivery tool of a bone anchoring device having a port for insertion of a filling material.

FIGS. 35A-35B are side views of a bone anchoring device when placed inside a bone and being in a collapsed state (FIG. 35A) and an expanded state (FIG. 35B).

FIGS. 36A-36D are a cut view (FIG. 36A) and a perspective cut view (FIG. 36B) of a bone with a bone anchoring device when placed inside the bone and being in a collapsed state; and a cut view (FIG. 36C) and a perspective cut view (FIG. 36D) of a bone with a bone anchoring device when placed inside the bone and being in an expanded state.

FIGS. 37A-37E show side, perspective and cross-sectional views of a delivery tool for inserting a bone anchoring device, according to an embodiment.

FIG. 38 depicts a cross-sectional view of a delivery tool coupled to a bone anchoring device, according to aspects of the present disclosure.

FIGS. 39A-39C depict side, perspective and cross-sectional views of a portion of a delivery tool when in an initial stage prior to the deployment of a bone anchoring device in a bone, according to aspects of the present disclosure.

FIGS. 40A-40B illustrate side and cross-sectional views of a portion of a delivery tool following the deployment of a bone anchoring device, in a bone according to an embodiment.

The following detailed description of embodiments of the invention refers to the accompanying drawings referred to above. Dimensions of components and features shown in the figures are chosen for convenience or clarity of presentation and are not necessarily shown to scale. Wherever possible, the same reference numbers will be used throughout the drawings and the following description to refer to the same and like parts.

### DETAILED DESCRIPTION OF EMBODIMENTS

Illustrative embodiments of the invention are described below. In the interest of clarity, not all features/components of an actual implementation are necessarily described.

Bone anchoring or fixation devices, such as surgical screws are essential components in many orthopedic procedures. Repair of bone fractures and injured ligaments, bone replacements, bone spacers implantations, and joint replacements typically require anchoring elements.

The present invention relates to bone anchoring devices and systems useful to strengthen and stabilize anchoring elements within bones, for example to strengthen and stabilize screws used to repair bone fractures, or screws that anchor surgical wires in the treatment of bone ligaments. The present invention pertains to bone anchoring surgical devices and systems and the uses thereof in the treatment of various orthopedic indications, such as, bone fractures repairs, muscle, ligament, and/or tendon repairs, bone replacements, treatments related to osteoporosis, osteoarthritis, and bone spacing.

The herein bone anchoring devices include a deformable or expandable structure (e.g., a mesh, and/or expandable wings) coupled to a bone anchor body. The deformable structure provides enhanced stability, an increased fixation surface, and improved anchoring capability. Furthermore, the device significantly enhances the osteointegration potential between the bone and the bone anchoring device.

In an embodiment of the invention, the deformable or expandable structure is movable between a collapsed, non-expanded state and an expanded state. In some embodiments of the invention, the deformable or expandable structure, when in an expanded state, bulges laterally with respect to the bone anchor body. In some embodiments of the invention, the deformable or expandable structure, when in an expanded state, has a diameter being larger than the diameter of the bone anchor body.

In some embodiments, the deformable structure in the expanded configuration is used to strengthen bone screws, bone anchors or other fixation devices.

In some embodiments, there are provided bone anchoring devices configured as expandable bone prostheses that can be deployed as replacements for removed bones or parts of bones. In some embodiments, there are provided deformable bone anchoring devices configured as bone spacers that connect adjacent bones.

The present invention pertains to deformable bone anchoring devices and systems that primarily include a deformable structure and a bone anchor body coupled to each other, wherein the expandable structure is configured to expand, optionally laterally/radially bulge, after expansion thereof within an inner body position.

As used herein the term “deformable structure” pertains to a structure designed to change its shape in response to an applied force, or pressure. Such deformation may be reversible, returning to the original shape when the force is removed or when a reverse force is applied. Such deformation may be permanent, retaining the new shape after deformation. In an exemplary embodiment, the deformable structure comprises deformable wings.

In some embodiments, the deformable structure is an “expandible structure”, i.e., a structure designed to increase in size, volume, or one or more dimensions thereof, when subjected to an applied force, or pressure, such as mechanical tension, compression, or inflation. In an exemplary embodiment, the expandable structure comprises an expandable mesh.

In one or more embodiments, the terms “deformable structure” and “expandable structure” are interchangeable and refer to a structure that can at least deform/expand radially in a soft tissue or a rigid (bone) tissue or a lumen in the body, e.g., a bone lumen. The deformable structure after expansion thereof may be hollowed and may be mechanically deformed/expanded or deformed/expanded by filling thereof with filler materials (e.g., fluids, semi-solid or solid materials).

Advantageously, the deformable structure may have a rounded shape, or a cylindrical shape, or a conical shape. The shape of the deformable structure may optionally adapt the shape and/or size of a bone, or a bone space being treated. The deformable structure may be porous and/or hollowed and may allow invasion of bone cells therethrough and/or growth of the bone cells therein. In one embodiment, the expandable structure is flexible, yet resists radial forces exerted thereon.

The deformable structure may be made from an elastic material that allows expansion of the structure following deployment thereof and at the same time resistance to radial forces of the bone.

Advantageously, the deformable structure is manufactured from a biocompatible material. In an exemplary embodiment, the deformable structure is made from a metallic material or combination of metals. Optionally, the deformable structure is made from shape memory alloy and may comprise nickel-titanium (a.k.a. NiTi, nitinol). The shape memory alloy (SMA) may comprise other metals, for example, the shape memory alloy may be an iron-based or a copper-based alloy. The SMA may comprise a combination of at least two metals selected from: zinc, copper, gold, iron, titanium, and nickel. The SMA may be based on NiTi alloy and may further include one or more additional alloying elements, including, but not limited to Al, Ag, Au, Cu, Fe, Ga, Ir, Nb, Pd, Pt, Rh, Ta5 or W. Optionally, the deformable structure is made from silicon, or a shape memory polymer, such as, (meth)acrylates, polyurethanes, and blends of polyurethane and polyvinylchloride.

In an exemplary embodiment, the deformable structure is an expandable structure comprising a mesh having a net like structure. The mesh may be made from a flexible material that allows expansion thereof under certain conditions. Advantageously, the mesh may have a rounded shape, or a cylindrical shape, or a conical shape. The shape of the mesh may optionally adapt the shape of a bone, or a bone space being treated.

The mesh may be made from a metallic material or combination of metals. Optionally, the mesh is made from shape memory alloy and may comprise nickel-titanium (a.k.a. NiTi, nitinol).

In an exemplary embodiment, the deformable structure comprises wings configured to expand laterally.

In an exemplary embodiment, the wings are made from an alloy.

As used herein the term “bone anchor body” refers to a distal or a proximal portion of the herein bone anchoring device. The bone anchor body may be an element that serves to hold an object firmly in the bone or may be attached to such element. Various bone anchor bodies are contemplated such as hollowed or non-hollowed bone screws, bone pins, bone screws with circular threads, or with diagonal threads, or with expandable gripping arms.

In some embodiments, the deformable structure is connected to a distal and/or a proximal bone anchor body. The bone anchor body can be hollow, enabling passage therethrough of the deformable structure. When using a hollow anchor body, in some cases it is possible to place surgical wires or coils through the cavity of the anchor body that can be used for suturing, and/or to strengthen and shape the deformable structure.

In some embodiments, one or more portions of the bone anchoring device, e.g., the deformable structure, and the bone anchor body and/or the suture wires may be made of one or more of the following biocompatible materials: nitinol, stainless steel, cobalt chrome, titanium, bio-absorbable materials, polymers (e.g., Polyetheretherketone (PEEK), Polylactic Acid (PLA) and Polyglycolic Acid (PGA), Polycaprolactone (PCL), Polydimethylsiloxane (PDMS), and Polyurethane) and the like.

In some embodiments, the deformable structure and/or bone anchor body is connected to surgical wires such as sutures or polymeric/metallic wires. In some embodiments, the suture wires pass inside the deformable structure and/or bone anchor body. In some embodiments, the suture wires pass outside of the deformable structure and/or bone anchor body.

In some embodiments, one or more portions of the bone anchoring device, e.g., the deformable structure, the bone anchor and/or the suture wires may be covered or coated by a covering/coating external layer made of, for example, expanded polytetrafluoroethylene (EPTFE) or other polymer layer.

In some embodiments, one or more portions of the bone anchoring device has a shape configured to have similar dimensions and/or shape as the treated bone or treated body tissue or body space. The shape of the deformable structure after deployment thereof within the bone may be, for example, spherical, cylindrical, circular, rounded, elliptic, or conical.

In exemplary embodiments, the deformable structure, may, for example, when implanted into a wrist or foot, adapt to the size and/or shape of the wrist or foot bone. Further, the deformable structure, may be expanded to dimensions larger than a drilled canal in the bone and not only fill the canal void, but also further expand laterally presenting larger dimensions, i.e., by pressing against the side walls of the drilled canal. Advantageously, bone tissue may grow and cover or integrate into the deformable structure, resulting in additional stabilization in the bone.

Advantageously, the device may be used in minimally invasive endoscopic surgical procedures. In some embodiments, the surgical procedure applicable for the present device includes drilling a canal through or within a bone, anchoring the device or a portion thereof in the target body position, and expanding the deformable structure (which may be self-expanded or proactively expanded) by mechanical forces, e.g., push forces, or by injecting filler materials, such as fluids, or solid particles.

In some embodiments, the deformable structure is hollow and enables introduction of filler materials like bone cement, autologous bone, or bone substitutes.

In some embodiments, the surgical procedure includes one or more of the following method steps: in a minimally invasive approach, preferably using an endoscope, drilling a hole at an anchoring site (the hole can be between 0.5-5 mm in diameter, but is typically up to about 8 mm), preferably through a working channel of the endoscope; introducing the bone anchor body or tip into the bone; expanding the deformable structure by either using a self-expanding element or by applying expansion means; and connecting surgical wires to the bone or body tissue.

In some embodiments, the suture wires can be anchored to a target bone or a nearby bone using the following technique: drilling at least one hole inside the target bone and using suture wires that pass through the hole in the drilled bone to make a tight anchor that holds the wires in the anchored bone.

In some embodiments, suture wires of nearby anchors can be secured to a bone using the herein bone anchoring device by pulling the suture wires and coupling them to the herein bone anchoring device and inserting the bone anchoring device through the drilled lumen to create a secure anchor that holds the wires firmly in place.

In some embodiments, the bone anchoring device is configured to connect broken bones. Specifically, the bone anchoring device may be used for anterior screw fixation of the cervical spine, or for bone fracture repair, such as the repair of long bones (e.g., the clavicle bone, or the fibula bone), or for repair of osteotomies.

In some embodiments, the bone anchoring device is used as a bone replacement. Specifically, the bone anchoring device may be used as a bone prosthesis that can replace a defective bone, such as a defective trapezium caused by rheumatoid arthritis, a traumatic arthritis, osteoarthritis, or post fracture deformation of the bone.

In some embodiments, the bone anchoring device is configured to strengthen and stabilize a bone anchor (e.g., screw or nail). For example, the device may be used to repair spinal cord fractures, or osteoporotic vertebrae. For example, the device can be used in cases of spinal tumor metastasis, for use at spinal levels where the structural integrity of the spine is not severely compromised. Additionally, the bone anchoring device may be used as an anchor stabilizer intended for use as a suture or tissue fixation device in any of the foot, ankle, knee, hand, wrist, elbow, shoulder, and hip.

Exemplary applicable elbow medical indications include biceps tendon reattachment/tear repair, ulnar or radial collateral ligament reconstruction, and lateral epicondylitis repair.

Exemplary applicable shoulder medical indications include rotator cuff repair, bankart repair, SLAP lesion repair, biceps tenodesis, acromio-clavicular separation repair, deltoid repair, capsular shift or capsulolabral reconstruction.

Exemplary applicable hand/wrist medical indications include: scapholunate ligament reconstruction, carpal ligament reconstructions, repair/reconstruction of collateral ligaments, repair of flexor and extensor tendons at the PIP, DIP, and MCP joints for all digits, and digital tendon transfers.

Exemplary applicable foot/ankle medical indications include lateral stabilization, medial stabilization, Achilles tendon repair, metatarsal ligament repair, hallux valgus reconstruction, digital tendon transfers, and mid-foot reconstruction.

Exemplary applicable knee medical indications include medial collateral ligament repair, lateral collateral ligament repair, patellar tendon repair, posterior oblique ligament repair, iliotibial band tenodesis, and joint capsule closure.

Exemplary applicable hip medical indications include capsular repair and acetabular repair.

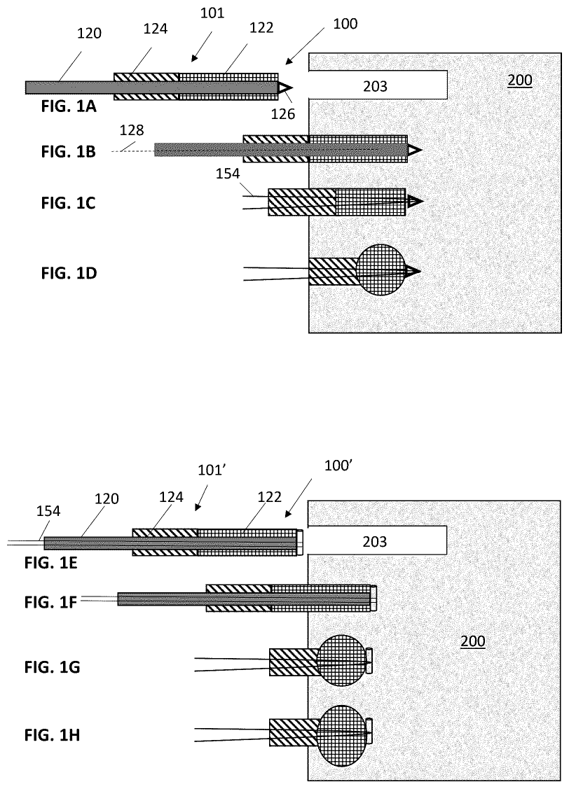

FIGS. 1A-1D show schematic side views of a bone anchoring surgical system 100, in various stages of implementation, in accordance with embodiments of the present invention. This embodiment is particularly useful for stably anchoring a bone, for example, for stably anchoring a bone screw in surgical ligament repair procedures, such as a rotator cuff repair.

The bone anchoring surgical system 100 includes an expandable bone anchoring device 101 and a delivery tool 120 (also referred to as delivery tool shaft) configured to deliver device 101 to a target inner body position and optionally to activate device 101.

Device 101 includes an expandable structure, illustrated herein as a flexible mesh 122 which is generally cylindrical in shape in its initial non-expanded state (FIGS. 1A-1C); and a bone anchor body 124. Device 101 may further include a suture wire coupler such as front eyelet 126 that allows passing therethrough suture wires 154. Suture wires 154 may be used to suture an injured bone ligament, such as a rotator cuff. Initially, delivery tool 120 is located along a longitudinal axis 128 of device 101, internally and co-axially with respect to mesh 122 and bone anchor body 124. Eyelet 126 is used to allow insertion of a wire, such as a surgical wire, therethrough, which can be used for various applications, e.g., for suturing (e.g., suturing a ligament), or bring two parts of a fractured bone together, or to evaluate the bone anchoring strength of device 101 (by externally stretching the wire). Eyelet 126 is located at the front of device 101, mesh 122 is disposed behind eyelet 126; and bone anchor body 124 is located behind and adjacent mesh 122 in a manner whereby bone anchor body 124 can press on the mesh. Anchor body 124 may be connected to mesh 122 by various means, e.g., by fusion, by crimping, by connecting loop(s) and the like.

In FIG. 1A, device 101 is about to be inserted in a bone 200, which has been drilled into to provide a lumen 203 for insertion of device 101. In FIG. 1B, device 101 has been partially inserted into bone 200 to the extent where mesh 122 is fully disposed in lumen 203 of bone 200. In FIG. 1C, device 101 has been further inserted into bone 200 to the extent where bone anchor body 124 is partially disposed in lumen 203 of bone 200. Anchor body 124 may be inserted via a striking or knocking action, optionally by a maneuvering action of delivery tool 120, although alternative insertion actions are contemplated, such as a screwing action. Optionally, withdrawal of delivery tool 120 and release thereof from device 101 allows mesh 122 to be released from its initial non-expanded configuration.

In FIG. 1D, device 101 has been completely inserted into bone 200 wherein bone anchor body 124 is fully disposed in lumen 203 of bone 200. Insertion of bone anchor body 124, for example by external striking action (optionally by delivery tool 120), pushes mesh 122 inward causing the mesh to bulge laterally and entirely fill the adjacent space in lumen 203 thereby applying lateral forces on the walls of lumen 203, and strengthening the anchoring effect. Continued pushing of bone anchor body 124 toward mesh 122 causes the mesh to further laterally bulge within lumen 203.

The surgical process, i.e., method of implementing the surgical system described with reference to FIGS. 1A-1D includes the following steps: (a) drilling into the bone to form lumen 203 that is dimensioned to receive mesh 122 and bone anchor body 124 of device 101; (b) inserting mesh 122 into lumen 203 in the target bone; (c) inserting bone anchor body 124, by striking and/or swiveling of delivery tool 120, into the bone (i.e., the walls of lumen 203) thereby pressing on mesh 122, which causes the mesh to bulge laterally against the walls of lumen 203. The above steps (a)-(c) may be repeated with anchoring yet another one or two devices 101 and thereby suturing an injured rotator cuff. The surgical suture wires 154 pass through and out from the bone anchor body 124 and are thereby stably attached to a bone, which allows stably suturing a ligament.

FIGS. 1E-1H show schematic side views of bone anchoring system 100′ having a surgical device 101′, in various stages of implementation, in accordance with embodiments of the present invention. Surgical device 101′ is similar to device 101 with the exception that surgical suture wires 154 pass through the device 101′ from an internal position thereof. In this configuration, suture wires 154 are connected to an internal position in a distal end of the device and extend internally to the proximal end of the device 101′ and beyond thereof. Alternatively, suture wires 154 can be slidable and are disposed about a pin in an internal position in a distal end of the device and extend internally to the proximal end of the device 101′ and beyond.

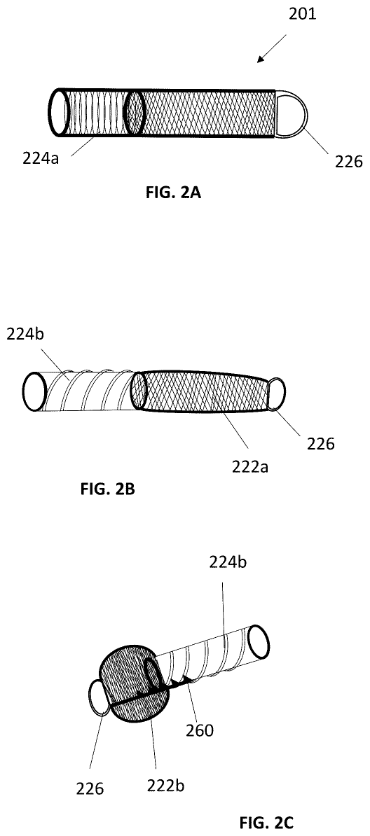

FIGS. 2A-2C show an expandable bone anchoring device 201 in various forms and configurations in accordance with embodiments of the present invention. Device 201 includes an expandable structure, illustrated herein as a flexible mesh 222 which is generally cylindrical in shape in its initial non-expanded state. Mesh 222 is connected to any of a variety of bone anchor bodies, such as bone anchor body 224a (FIG. 2A) or bone anchor body 224b (FIGS. 2B, 2C) and optionally includes a front eyelet 226. Anchor body 224b has diagonal threading (FIGS. 2B and 2C) for insertion thereof by swiveling, although knocking insertion may also be applicable. Anchor body 224a has a series of circular threads (FIG. 2A), suited for insertion by a striking action.

Various types of mesh 222 are contemplated, such as a mesh manufactured from a shape memory material (e.g., nitinol) or the like. Mesh 222 may have various shapes, such as a cylindrical shape (e.g., mesh 222a) or a rounded shape (e.g., mesh 222b). In some embodiments, device 201 is movable between a non-expanded state (FIGS. 2A and 2B), which is applicable mainly during delivery of device 201 to the target bone; and an expanded state (FIG. 2C) after deployment in the target bone. Device 201 may include a locking mechanism configured to lock the mesh 222 in the expanded configuration. Various forms of locking mechanisms are contemplated such as zip ties 260 illustrated in FIG. 2C, or other forms including hooks, locking teeth, pins, ratchet teeth, threads, and the like. Device 201 may be useful for repairing an orthopedic indication, such as a rotator cuff.

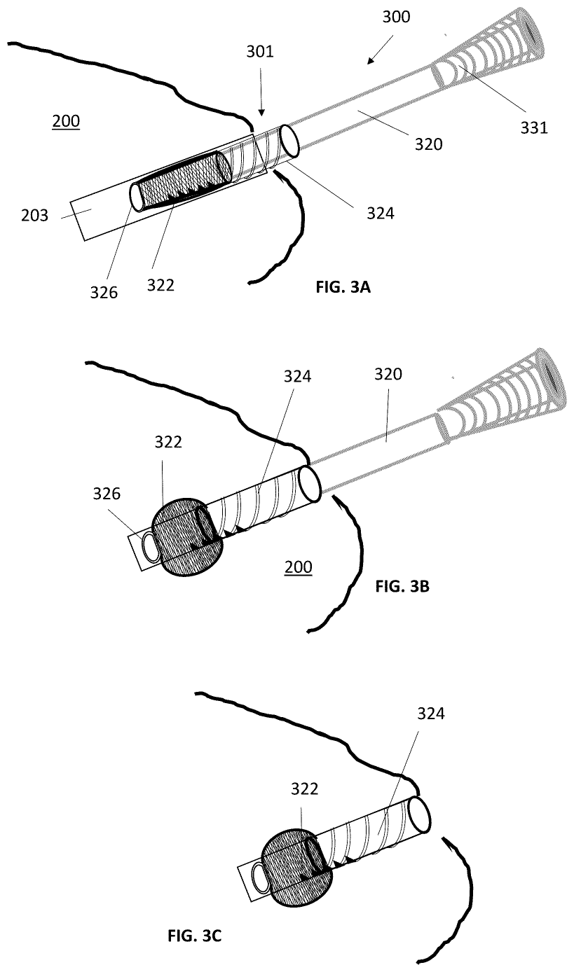

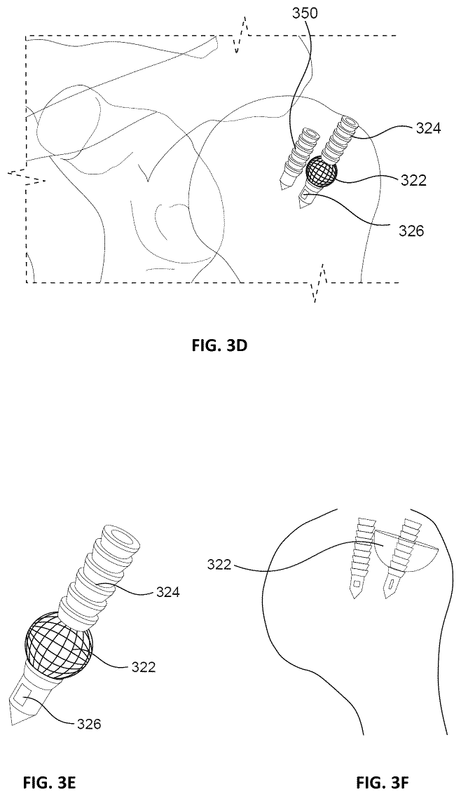

FIGS. 3A-3F schematically show a bone anchoring surgical system 300 with an expandable bone anchoring device 301 in accordance with embodiments of the present invention that may be used to repair a rotator cuff. Device 301 uses or includes a delivery system in the form of a delivery tool shaft 320 with a funnel-shaped maneuvering element 331 at its free proximal end. At its other distal end, delivery tool shaft 320 is coupled to bone anchor body 324, which is located adjacent mesh 322, as noted above. For repairing a ligament (e.g., a rotator cuff), device 301 also includes a front eyelet 326 through which a suture wire can be inserted (not shown) to allow suturing a ligament, such as the rotator cuff. The device 301 allows stably anchoring the suture wire via the bone anchoring device 301.

FIG. 3A shows device 301 connected to a delivery tool shaft 320 with the device 301 inserted into bone 200 to the extent where bone anchor body 324 is partially disposed in lumen 203. Insertion of bone anchor body 324 pushes on mesh 322 whereby the mesh bulges laterally thereby strengthening and stabilizing the anchoring action. FIG. 3B shows anchor body 324 after it is inserted into lumen 203, with mesh 322 expanded, as noted above.

FIG. 3C shows device 301 disconnected from delivery tool shaft 320.

FIGS. 3A-3C illustrate an exemplary surgery process in accordance with embodiments of the invention for repairing a rotator cuff. The method includes: (a) drilling into the humerus bone to form lumen 203 dimensioned to receive device 301; (b) inserting eyelet 326 with a suture wire and mesh 322 into lumen 203; (c) inserting bone anchor body 324 into lumen 203 of the humerus bone, for example by striking or swiveling the delivery tool shaft 320 thereby pressing on mesh 322, causing mesh 322 to bulge laterally against the walls of the lumen; and (d) releasing the delivery tool shaft 320. Further steps of inserting one or more screws 350 (FIG. 3D) or bone anchor bodies with a mesh (the herein device 301) with suture wires passing therethrough or connected thereto are contemplated to allow repairing an impaired rotator cuff. The surgical wires that pass through each of those anchors are now stably anchored to the bone due to the bulging mesh 322 that stably holds bone anchor body 324 in the bone and can then be sutured to the rotator cuff and used to repair it.

FIG. 3D shows device 301after a repair surgery of a rotator cuff.

FIG. 3E is a perspective view of device 301 that includes an anchor body 324; a mesh 322 configured to laterally bulge following implantation thereof within a bone canal; and a front eyelet 326 for inserting a surgical wire therethrough that can suture a ligament, such as a rotator cuff.

FIG. 3F illustrates device 301 wherein the mesh 322, when pushed by anchor body 324 or a delivery tool shaft 320, is folded internally forming a double walled mesh having an umbrella-like structure that adds strength and rigidity to mesh 322 when in the expanded state.

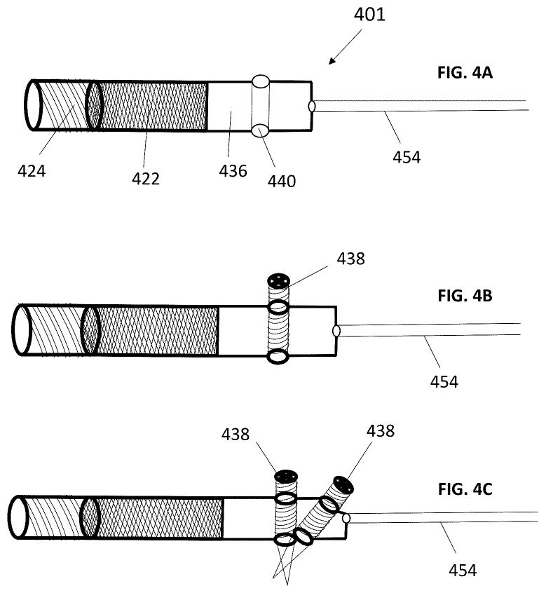

FIGS. 4A-4C show an expandable bone anchoring device 401 in accordance with embodiments of the present invention that may be used to repair bone fractures, such as fractures in the clavicle or fibula.

Expandable bone anchoring device 401 includes one or more auxiliary bone fixation members, such as bone screws 438. Bone screws 438 may be disposed at a different angle with respect to distal bone anchor body 424. Distal bone anchor body 424 is disposed at the distal end of device 401; and mesh 422 is disposed behind the bone anchor body 424. Behind mesh 422 is an elongated bar 436 with a generally transverse (auxiliary) bone screw 438 therein, in particular within a transverse tunnel 440 of the elongated bar 436. Device 401 may further include suture wires 454 connected to the distal end of anchor body 424 and/or mesh 422, and/or elongated bar 436 and configured to bring both parts of the fractured bone together.

FIG. 4C illustrates an embodiment in which device 401 has two bone screws, such as a first transverse bone screws 438; and a second (auxiliary) bone screw 438 disposed at a different angle.

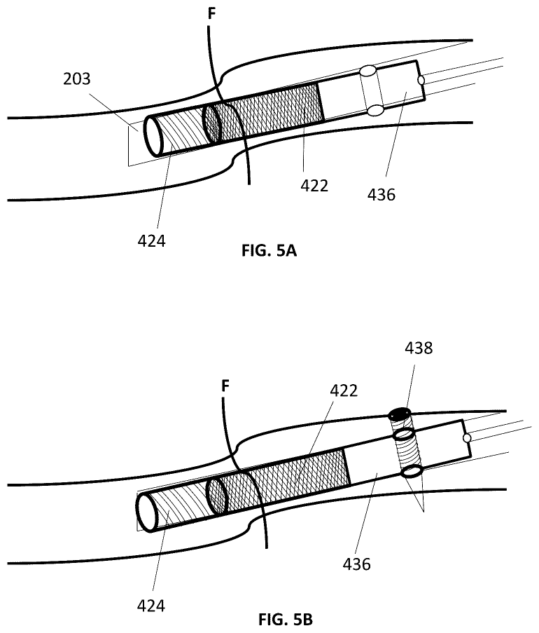

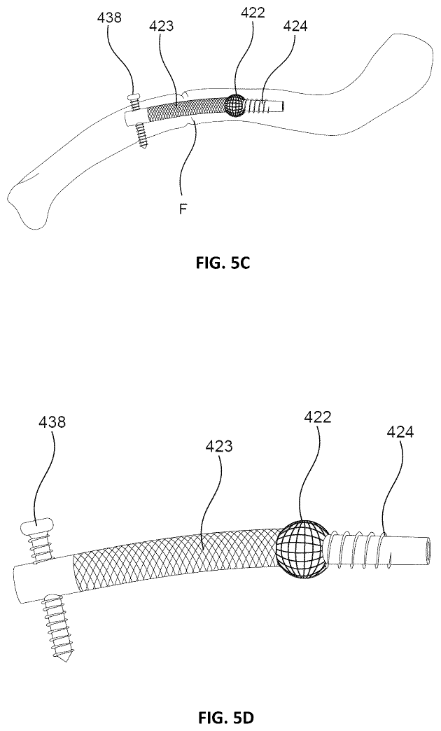

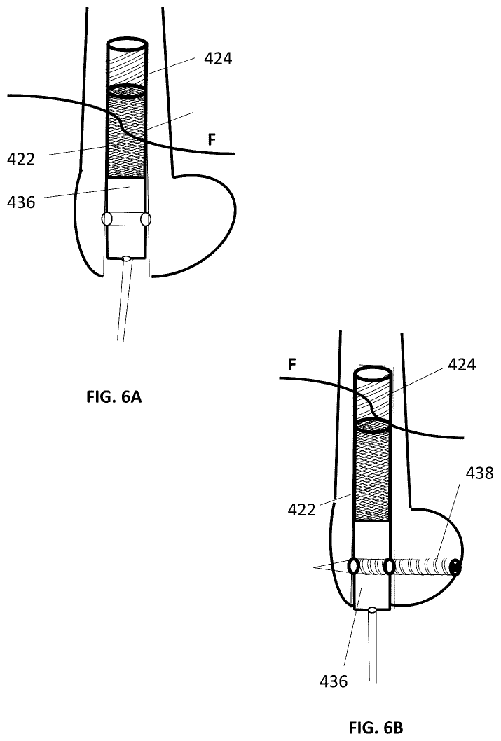

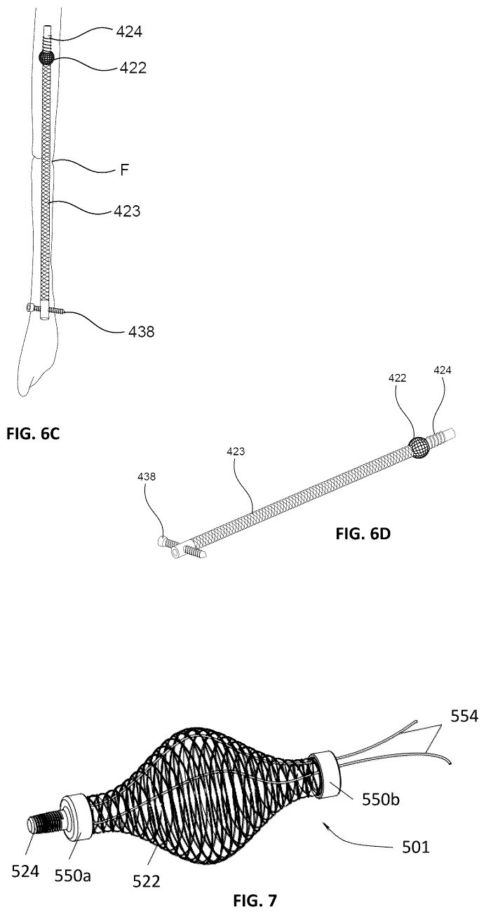

FIGS. 5A-5D and FIGS. 6A-6D show device 401 being implemented in a drilled-out portion (lumen 203) of a bone, for repairing bone fractures, such as clavicle (FIGS. 5A-5D) or fibula (FIGS. 6A-6D) fractures. Mesh 422 may be constructed with a shape memory metal, such as nitinol. Mesh 422 may have a distal rounded shape 422 following expansion thereof at the site of implantation and may continuously extend proximally and present a cylindrical mesh extension 423 such to bridge the site of bone fracture F. The cylindrical mesh extension 423 may be constructed from a braided or a non-braided nitinol, or from other braided or non-braided metal or metal alloys. In one embodiment, the cylindrical mesh extension 423 is made of a material that is more rigid than the distal mesh 422. Optionally, the cylindrical mesh extension 423 has a length that corresponds to the length of the fractured bone. One prominent advantage of the herein device 401 is that contrary to current nails, the cylindrical mesh extension 423 which is made from a flexible material, enables micromovements of the fractured bone and those micromovements induce and accelerate bone recovery.

The surgical process, i.e. method of implementing the device described with reference to FIGS. 4-6, includes the following steps: (a) drilling into the fractured F bone to form lumen 203, which is dimensioned to receive device 401; (b) inserting anchor body 424 and mesh 422 into the lumen in the bone; (c) inserting elongated bar 436 into the bone (i.e. into the lumen), for example by striking or swiveling, thereby pressing on mesh 422, causing mesh 422 to bulge laterally against the walls of the lumen; and (d) screwing the one or more bone screws 438 into the bone at tunnel 440 (FIGS. 5B and 6B).

FIG. 7 shows an expandable bone anchoring device 501 in accordance with embodiments of the present invention that may be used to replace bones, or fill the space of a missing bone, such as the trapezium bone in the hand. In some medical conditions, in which there is a defective bone, such as a defective trapezium, there is a benefit in inserting a bone replacement element. Such replacement elements can help maintain the normal bone and joint configuration and function by retaining the normal bone function and provide support to the other nearby bones. Further examples of bones that can be surgically removed and replaced by device 501 include the lunate bone and the mid foot bones (not shown).

Device 501 includes a bone anchor body 524. Bone anchor body 524 is located at the distal end of device 501 and is followed by a mesh 522, which in this indication, operates as a bone-replacement member, illustrated as an expandable mesh 522. On either end of bone-replacement mesh 522 are mesh-expansion elements, for example, a distal annular mesh-expansion element 550a, to which bone anchor body 524 is attached; and a proximal annular mesh-expansion element 550b, which may have a plug-like configuration, as illustrated, and which attach mesh 522 at both ends thereof. One or more suture wires 554 (two are illustrated) are connected to and expand from the interior of distal mesh-expansion element 550a. Wires 554 pass through mesh 522 and also through proximal annular mesh-expansion element 550b.

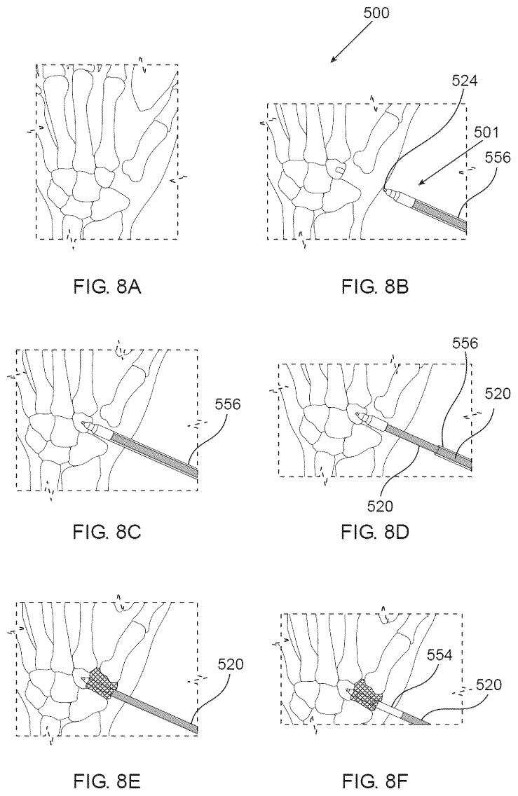

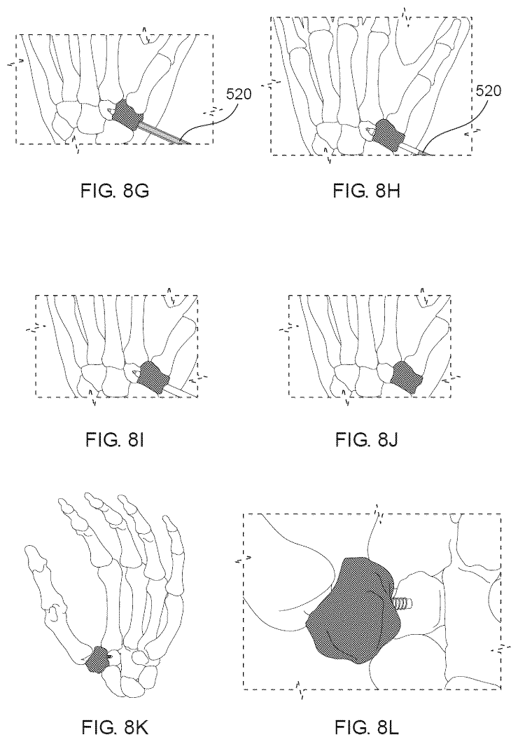

FIGS. 8A-8L illustrate a bone anchoring surgical system 500 that includes device 501 implemented in a trapezium replacement procedure. After removal of a defective/injured trapezium bone (FIG. 8A), bone anchor body 524 is inserted into the trapezoid bone of the hand, which may be proceeded by drilling a canal into the trapezoid bone. Bone anchor body 524 is inserted to an appropriate depth, which takes into account both secure anchoring and the location of mesh 522 relative to the surrounding bones (FIGS. 8B-8C). After that, a sheath 556, within which device 501 is initially placed, is retracted (FIG. 8D). Mesh 522 is then self-expanded to suitably fill the space left by the removed trapezium bone (FIG. 8E). Delivery tool 520 is then retracted (FIG. 8F), optionally by releasing connecting wires 554. In an optional step (FIG. 8G), before delivery tool 520 is retracted, mesh 522 is filled with a filler material, e.g., bone cement, or ligament, or any other applicable filler material(s). Delivery tool 520 is then removed (FIGS. 8H and 8I) and connecting wires 554 are cut and/or used for suturing, and/or removed. It is to be noted that mesh 522 may be expanded by the filler material, such that after sheath 556 is retracted, the mesh is still in the non-expanded state or is partially extended and is fully expanded following filling thereof with the filler material.

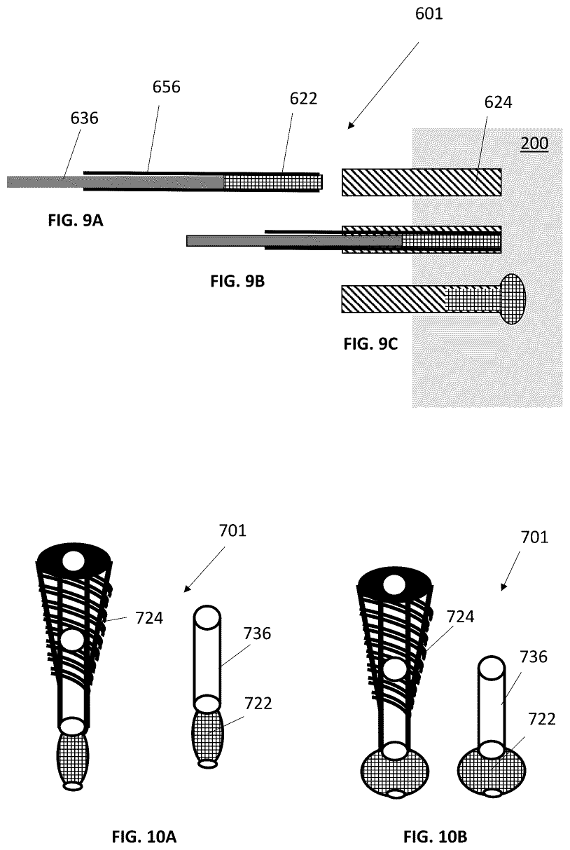

FIGS. 9A-9C show an expandable bone anchoring device 601 in accordance with embodiments of the present invention that may be useful for spine (pedicle) surgery. Device 601 includes a hollow bone anchor body 624 (e.g., a spine screw) and an elongated mesh 622, configured and dimensioned so that the mesh can pass through the hollow anchor body 624 in a non-expanded state. Mesh 622 is also configured and dimensioned so that it can be inserted into hollow bone anchor body 624. Device 601 may optionally include an elongated bar 636 attached thereto and which may optionally be implanted along with device 601 and/or act as a delivery tool. Device 601 may also include a sheath 656 that aids in the delivery of the mesh 622 and optionally holds mesh 622 in the non-expanded state.

FIG. 9A illustrates hollow bone anchor body 624 inserted into bone 200. FIG. 9B illustrates elongated bar 636 inserting mesh 622 into the hollow portion of hollow bone anchor body 624. In FIG. 9C, the distal end of mesh 622 has been pushed passed the distal end of hollow bone anchor body 624; sheath 656 has been retracted; and elongated bar 636 has been removed.

FIGS. 10A-10B show an anchoring device 701 in accordance with embodiments of the present invention that may be useful for spine (pedicle) surgery. FIG. 10A shows a mesh 722 of device 701 in its non-expanded state; and FIG. 10B shows the mesh 722 in its expanded state. Mesh 722 is disposed at the distal end of device 701, which also includes a hollow bone anchor body 724, optionally having a frustoconical shape; and an intermediate elongated bar 736.

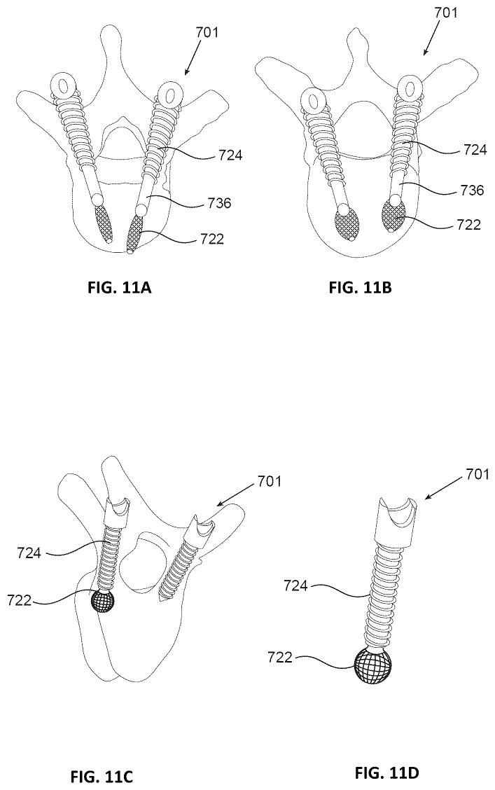

FIGS. 11A-11D illustrate bone anchoring device 701 being implemented. In FIG. 11A, device 701 is inserted in a vertebra; and in FIG. 11B, and 11C mesh 722 is expanded, which can be via any of the methods described above (i.e., self-expanded, expanded by filling thereof with a filler material, and/or expanded by push forces applied thereon). FIG. 11D is a perspective view of device 701 in an expanded state wherein the mesh 722 bulges laterally from anchor 724.

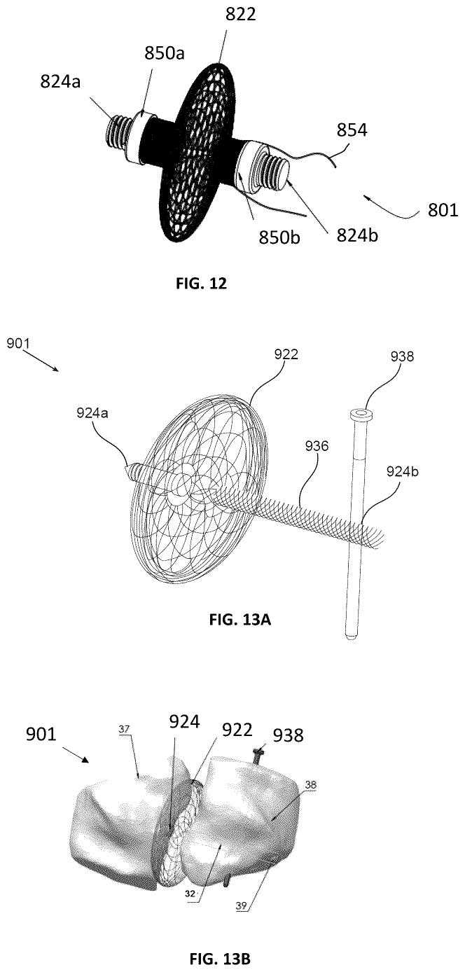

FIG. 12 illustrates another exemplary bone anchoring device 801 in accordance with embodiments of the herein invention. Device 801 may be implemented as an inter-bone spacer that includes a first (distal) bone anchor body 824a located at the distal end of device 801 and a second (proximal) bone anchor body 824b located at the proximal end of device 801. Between bone anchor bodies 824a and 824b is a mesh 822, which in this embodiment operates as a bone spacer that connects adjacent bones or replaces a missing bone. On either end of mesh 822 are mesh-expansion elements 850 configured to hold and attach mesh 822 to bone anchor bodies 824. One or more suture wires 854 (two are illustrated) are connected to and configured to aid in evaluating the strength of fixation of the bone anchor body and mesh in the bone. Suture wires 854 may be disposed outside mesh 822 and may extend along device 801 from anchor 824a and/or from distal mesh-expansion element 850a. Alternatively, suture wires 854 pass through device 801.

FIGS. 13A-13B illustrate yet another bone anchoring device 901 that may be implemented as a bone spacer. Device 901 includes a mesh 922 connected at a distal end thereof to a distal bone anchor body 924a that is used for anchoring to a distal bone 37. At the proximal end there is a proximal anchor body 924b that allows fixation to a proximal bone 38 via bone screw 938. The device 901 further includes an elongated bar 936 that is placed inside canal 32 in proximal bone 38 and bridges between mesh 922 and the proximal bone anchor body 924b. Elongated bar 936 may be made of various rigid materials, or from a resilient or semi-resilient mesh tube.

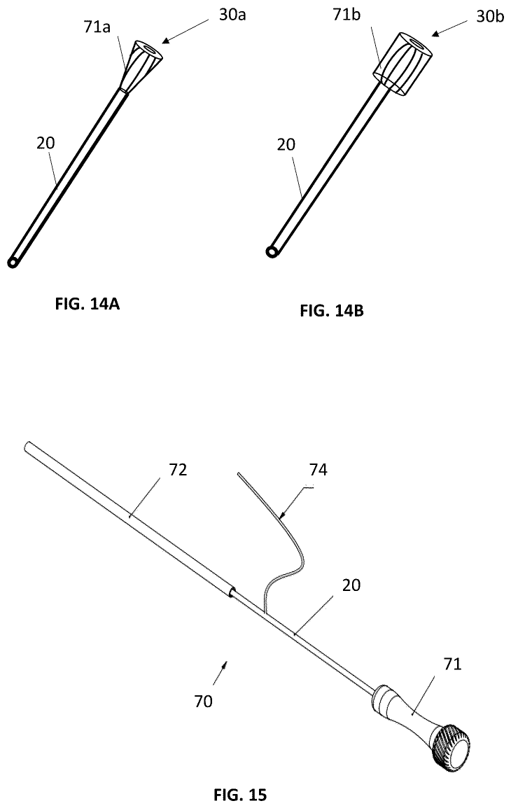

FIGS. 14A-14B illustrate exemplary delivery tools 30 for the present bone anchoring devices. FIG. 14A shows a delivery tool 30a including a delivery tool shaft 20 and rotation mechanism 71a that enables rotation of the device before full deployment. FIG. 14B shows a delivery tool 30b including delivery tool shaft 20 and a cylindrically knocking means 71b that enable knocking the bone anchor body onto a target bone. It is to be noted that rotation mechanism 71a also enables knocking thereon to facilitate anchoring the anchor to the target bone.

FIG. 15 illustrates a further exemplary delivery tool 70 for the present bone anchoring devices. Delivery tool 70 allows inserting the devices in minimally invasive approaches. Delivery tool 70 includes rotation mechanism 71 that enables rotation of the device before full deployment. Exemplary rotation mechanism 71 may include or be constituted, for example, by a knob.

Delivery tool 70 may include an external sheath 72 that holds the device in a crimped configuration and can be used for deployment and expansion of the devices in a target inner body position. External sheath 72 may be used also for retracting delivery tool 70. Delivery tool 70 may also include an internal delivery tool shaft 20 connected to the device (not visible) and to rotation mechanism 71. Delivery tool shaft 20 is used for both swiveling the device during anchoring and/or to push the device out of the external sheath 72, and/or to knock the device to insert it into the inner body position. In some embodiments, an injection tube 74 is used to inject filler material into the mesh.

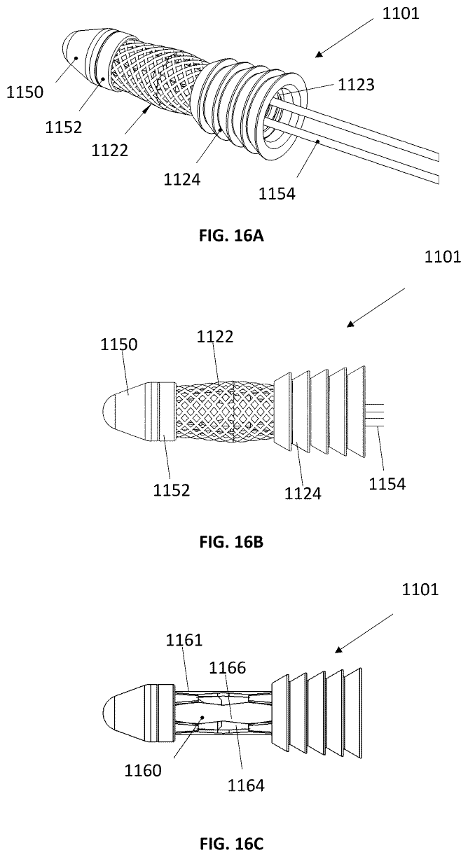

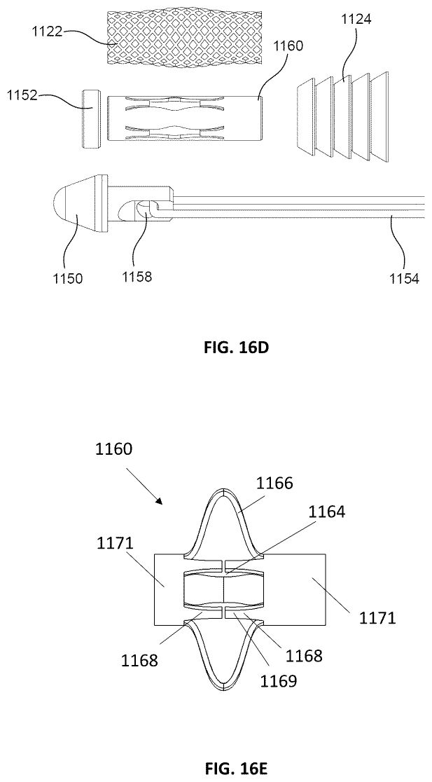

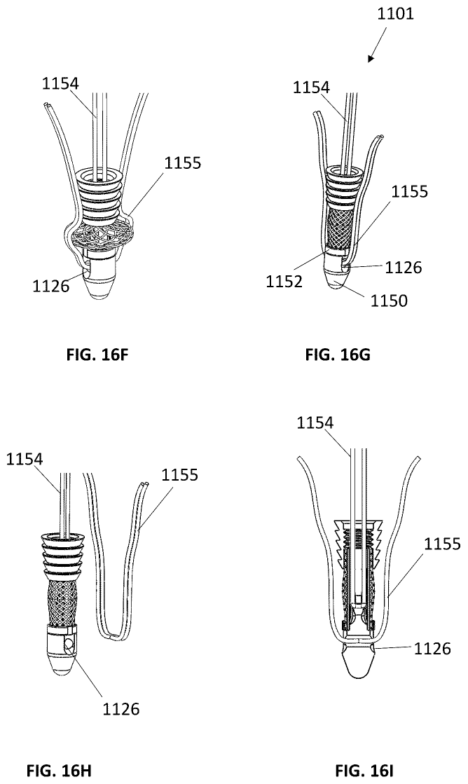

FIGS. 16A-16E show a bone anchoring device 1101 in accordance with embodiments of the present invention that may be used for example in shoulder arthroscopy procedures, such as of the rotator cuff tears repair.

FIG. 16A is an isometric view of bone anchoring device 1101 comprising mesh 1122. FIG. 16B is a side view of bone anchoring device 1101. FIG. 16C is a side view of bone anchoring device 1101 without a mesh 1122. FIG. 16D is an exploded view of bone anchoring device 1101. FIG. 16E is a side view of deformable structure 1160 of bone anchoring device 1101.

From FIGS. 16A-16B it can be seen that bone anchoring device 1101 may have a tubular like structure, which may be closed at a distal end of the device and open at a proximal end of the device 1101. Anchoring device 1101 may include a deformable structure 1160 without a mesh 1122. Anchoring device 1101 may include a mesh 1122 only. Anchoring device 1101 may include a mesh 1122, and a deformable structure 1160. The bone anchoring device 1101 may include a bone anchor body 1124, featured by an external thread or a ribbed element for fixation in cortex bone tissue. The bone anchoring device 1101 may be hollowed and have a central cavity 1123. The bone anchor body 1124 may be disposed at the proximal end of the device 1101. The bone anchor body 1124 may be disposed at any suitable angle with respect to mesh 1122 and the deformable structure 1160. The bone anchor body 1124 may be coupled to the bone anchoring device 1101 so that it extends in the same longitudinal line as the mesh 1122, the distal tip 1150 and the deformable structure 1160. At a distal end of the device 1101 is a device tip 1150. The tip 1150 may be closed at its distal extremity. The tip 1150 may be rigid. The tip 1150 may be coupled to a support ring 1152, which may be positioned at the proximal base of the tip 1150. Mesh 1122 may be suitably coupled to the bone anchor body 1124. The mesh 1122 may be a tubular length of mesh, which may be attached at its distal end to the support ring 1152 and at its proximal end to the bone anchor body 1124. The mesh 1122 may be disposed about the deformable structure 1160. The mesh 1122 may enclose at least part of the deformable structure 1160. The mesh may enclose the entire surface of deformable structure 1160 (FIG. 16C). The mesh 1122 may encircle at least part of the deformable structure 1160. The mesh may encircle the entire length of deformable structure 1160 (FIG. 16C). The mesh 1122 may have more than one mesh layer. Multiple mesh layers may improve bone fixation forces due to improved tissue growth within the meshes, thereby accelerating the integration between the implant and the inner bone tissue. Anchoring device 1101 may further include one or more suture wires 1154. The suture wires 1154 may be accommodated in the cavity 1123 of the anchoring device 1101. The suture wires 1154 may be surgical wires, suitable to suture body tissues, such as an injured tissue (i.e., a torn tendon).

As shown in FIGS. 16C-16E, the bone anchoring device 1101 may include an element, which is deformable 1160. The term ‘deformable’ as used herein may refer to a structure which may reversibly change shape, such as it may expand laterally and optionally reversibly collapse to the initial form. The deformable structure 1160 may be coupled at a distal end to the tip 1150 of the device 1101, and at a proximal end to the bone anchor body 1124 wherein the deformable structure 1160 is disposed between the device tip 1150 and the bone anchor body 1124. The suture wires 1154 may also be configured to be employed to facilitate deformation of the mesh 1122 and any other attached deformable structures 1160 of the anchoring device 1101. FIG. 16C shows the deformable structure 1160 without the mesh and when in a collapsed/non-expanded state. FIG. 16D shows an exploded view of at least some of the components employed in the anchoring device, such as the mesh 1122, the bone anchor body 1124, the support ring 1152, the deformable structure 1160 and the device tip 1150 and suture wires 1154 as described hereinabove. FIG. 16E shows the deformable structure 1160 without the mesh and when in an expanded state. Suture wire 1154 may extend from a suture wire coupler such as internal eyelet 1158 and pass-through deformable structure 1160 and bone anchor body 1124 and out of device 1101. Suture wire 1154 may be fixedly attached to eyelet 1158 or may be slidable about eyelet 1158.

In one non-limiting example such as shown in FIG. 16C, the deformable structure 1160 may feature a tubular body 1161 with a plurality of spaces (e.g., laser-cuts) 1164 accommodating deformable/expandable wings 1166. The spaces (e.g., laser-cuts) 1164 may be spaced apart along the periphery of the deformable structure 1160. Each cut 1164 may extend along the length of the deformable structure 1160. The spaces 1164 may have a length of from about 1 mm to about 30 mm. The spaces 1164 may have a maximal diameter of from about 0 mm to about 15 mm. The deformable structure 1160 may have any number of suitable wings 1166. The deformable structure 1160 may have from about two to about eight wings 1166, depending on the desired diameter and stiffness of the structure 1160. The deformable structure 1160 may be constructed from a material so that it is reversibly deformable and may be suitably deformed on application of a suitable force on the tubular body 1161. For example, when the deformable structure 1160 is pushed proximally (i.e., toward the physician) so that the tip 1150 is pulled in a direction towards the bone anchor body 1124, the wings 1166 of the deformable structure 1160 may be deformed into an expanded or contracted state, as shown in FIG. 16E. When a reverse force is applied it may adopt a collapsed or non-contracted/non-expanded state (as shown in FIG. 16C). The deformable structure 1160 may be made from a material such as, but not limited to titanium, nitinol and stainless steel. The deformable structure 1160 may include at least one stopper 1169 with structural stabilizing element comprising ribs 1168 to prevent the anchoring device 1101 from being overly deformed as shown in FIG. 16E. Each stopper 1169 may feature two ribs 1168. The stoppers 1169 may comprise two ribs 1168 extending from the two opposing base elements 1171 of deformable structure 1160. The ribs 1168 limit the extent of contraction of deformable structure up to the point where two opposing ribs 1168 come into contact. The rib 1168 may be rigid and may limit the deformable structure 1160 to contract up to a length of the space 1164, which is equivalent to twice or more the length of the rib 1168. The expandable mesh 1122 may be disposed so that it encloses the deformable structure 1160. The expandable mesh 1122 may be disposed so that it encircles the deformable structure 1160. The deformable structure 1160 may be configured to facilitate deformation of the shape of the mesh 1122, such as expanding the mesh. The deformable structure 1160 may also be configured to support an expanded mesh 1122, wherein the expanded mesh protrudes outwards, out of the periphery of the body of the bone anchoring device 1101.

FIGS. 16F-16I illustrate yet another exemplary embodiment of bone anchoring device 1101 which additionally includes a second suture wire coupler, in the form of eyelet 1126 that allows passing therethrough suture wires 1155 that are used to suture an injured tissue, such a torn tendon. Eyelet 1126 allows passing therethrough one or more than one (e.g., two or three, or four) suture wires 1155. Suture wires 1155 may be similar to wires 1154 and made from the same materials. Suture wires 1155 may be different from wires 1154 and made from different materials. Suture wires 1155 may be used to suture a tissue in addition to wires 1154. Alternatively, suture wires 1155 may be used to suture a tissue and wires 1154 may be used to facilitate the deformation of deformable structure 1160. Eyelet 1126 may be disposed in various locations, such as at the distal most end of bone anchoring device 1101, as illustrated for example in FIGS. 1A-1D, for anchoring device 101. Eyelet 1126 may be disposed proximally to tip 1150, such as shown in FIGS. 16F-16I, and located between tip 1150 and support ring 1152. As shown in FIG. 16F, when the bone anchoring device 1101 is in the expanded configuration wires 1155 pass along the outer periphery of the bulged configuration of mesh 1122 and thereby tightly fixated to the bone tissue. Advantageously, due to the superior fixation provided by the herein bone anchoring device 1101, less bone anchoring implants may be required per procedure.

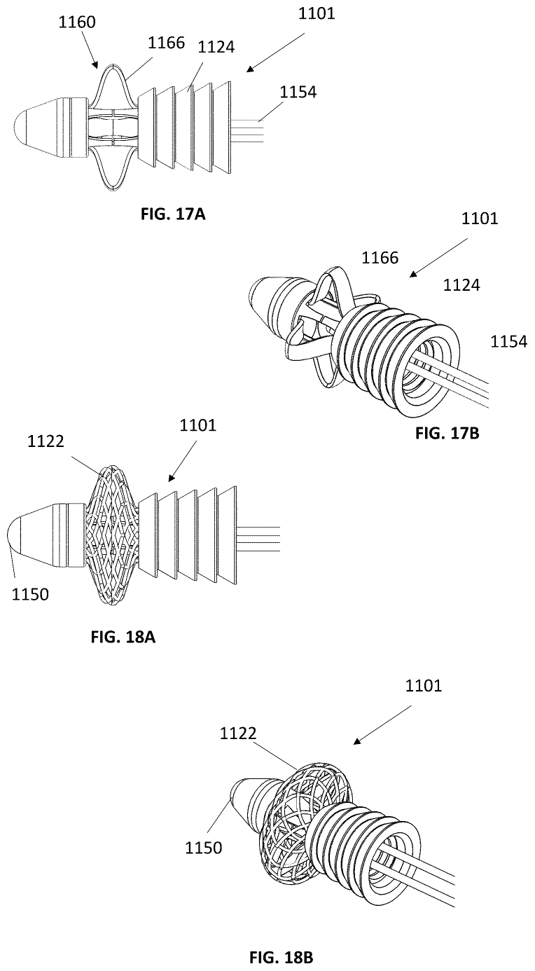

FIG. 17A is a side view of anchoring device 1101 without a mesh and when in an expanded state. FIG. 17B is an isometric view of anchoring device 1101 without a mesh and when in an expanded state. FIG. 18A is a side view of anchoring device 1101 with a mesh and when in an expanded state. FIG. 18B is an isometric view of anchoring device 1101 with a mesh and when in an expanded state. The wings 1166 of the anchoring device 1101 may be configured as any suitable structure which can protrude out of the periphery of the anchoring device and can reversibly collapse into the periphery of the bone anchoring device. The wings 1166 when in the extended configuration may form a triangular or concave like shape or any other suitable bulging shape. The wings 1166 may be configured to provide a support for a surrounding expanded mesh 1122 as shown in FIGS. 18A and 18B. The mesh 1122 may expand radially outwardly and may form a spherical mesh body or may have a balloon-like structure. The wings 1166 may be made from any suitable material, which has sufficient plasticity to reversibly bend to form a protruding wing, such as, but not limited to titanium, nitinol and stainless steel. The mesh 1122 of the anchoring device 1101 may be constructed with a shape memory metal, such as nitinol. The mesh 1122 may be constructed from a braided or a non-braided nitinol, or from other braided or non-braided metal or metal alloys. For example, the mesh 1122 may be made of a specially cut titanium tube that may form a mesh-like structure.

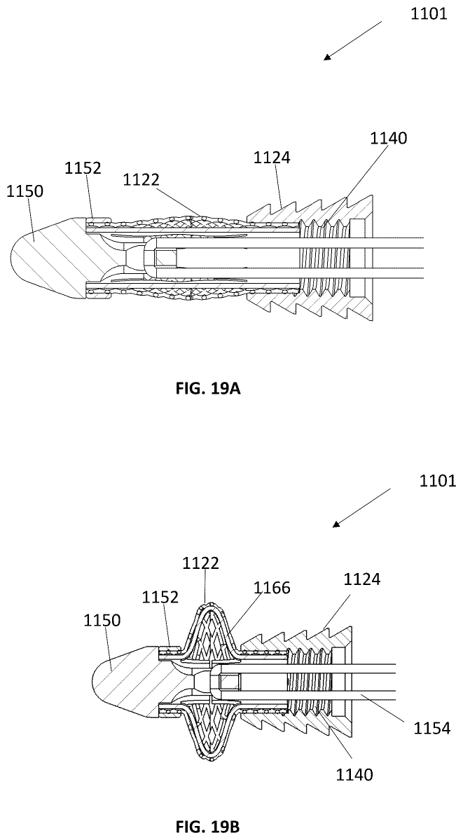

FIG. 19A is a cross section view of bone anchoring device 1101 in a collapsed state. FIG. 19B is a cross section view of bone anchoring device 1101 in an extended state. The bone anchoring device 1101 may adopt a plurality of configurations. In a first configuration, such as during insertion of the anchoring device 1101, the wings 1166 may be in a collapsed state and lie flat in the plane of the deformable structure 1160. In the first configuration, the deformable tubular structure 1160 may be in an undeformed configuration. The mesh 1122 may surround the deformable structure 1160 in a non-expanded flat conformation as shown in FIG. 19A. The mesh 1122 may be attached at a distal end to the tip support ring 1152 and at a proximal end to the bone anchor body 1124. In a second configuration, such as, but not limited to after the anchoring device 1101 has been inserted into a bone, the wires 1154 may be pulled proximally. The device tip 1150, which is coupled to the wires 1154 may be moved proximally to apply a force on deformable structure 1160 and the mesh 1122. The applied force may push the deformable structure 1160 into an expanded conformation and the wings 1166 may be pushed out of the deformable structure 1160 in an outstretched conformation and may protrude out of the periphery of the deformable structure 1160. The applied force on the mesh 1122 resulting from the pulling force on the anchoring device tip 1150 and the protruding wings 1166 may push the mesh to expand outwardly radially. The protruding wings 1166 may support the outer layer comprising of expanded mesh 1122 as shown in FIG. 19B. The mesh 1122 may form a spherical mesh extending radially out of the plane of the anchoring device 1101. An internal thread 1140 may be further extended from a proximal end of the anchor body 1124 to allow for an engagement with a retrieving tool as described in greater detail below. The internal thread 1140 in some embodiments may have a left-hand thread to ensure efficient coupling with a retrieving tool, as will be explained later herein.

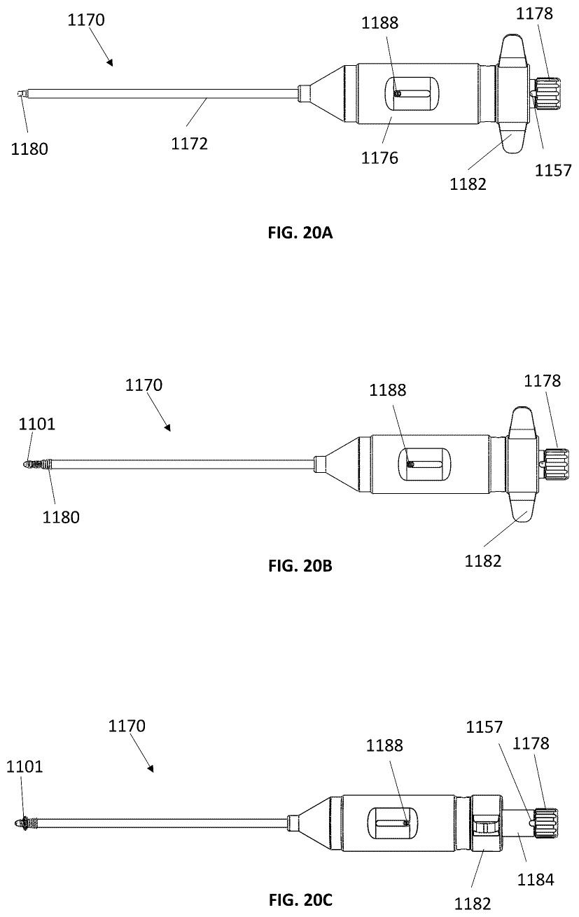

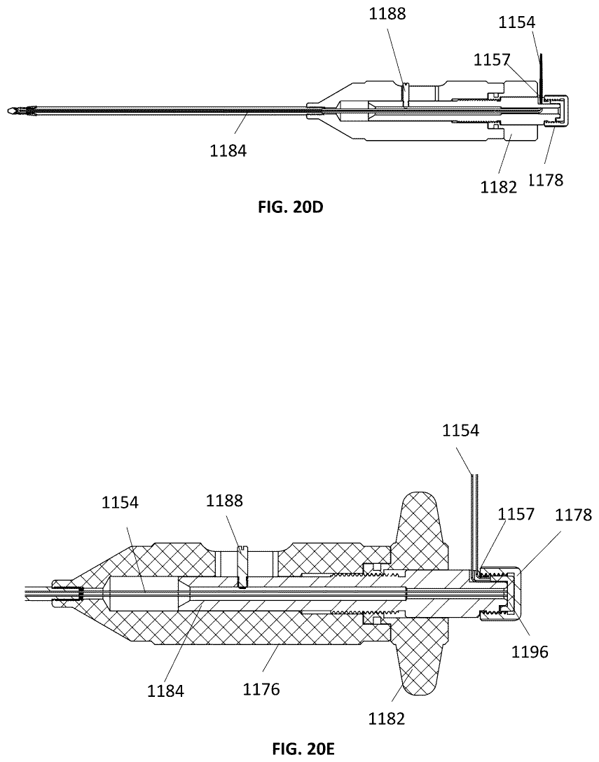

FIGS. 20A-20E show an exemplary delivery tool 1170 which may be used to insert a bone anchoring device 1101 in accordance with embodiments of the present invention. FIG. 20A is a side view of an exemplary delivery tool 1170 without the bone anchoring device 1101 attached to it. FIG. 20B is a side view of an exemplary delivery tool 1170 with the bone anchoring device 1101 attached to it. FIG. 20C is a side view of an exemplary delivery tool 1170 with the bone anchoring device 1101 attached to it when it is in the expanded state. FIG. 20D is a cross section view of an exemplary delivery tool 1170. FIG. 20E is a cross section view of an enlarged section of the handle 1176 of an exemplary delivery tool 1170.

The delivery tool 1170 essentially includes a delivery tool shaft 1172, and a delivery tool handle 1176. The delivery tool shaft 1172 is shaped and sized for insertion into the lumen of the bone to implant the bone anchoring device 1101 in the bone. A tip 1180 of the delivery tool shaft 1172 is configured to couple to the bone anchoring device 1101 of the present invention. The tip 1180 of the delivery tool shaft 1172 is configured to couple the proximal end of the bone anchor body 1124 of anchoring device 1101. The tip 1180 of the delivery tool shaft 1172 is configured to attach to the bone anchor body 1124 of the anchoring device 1101, as shown in FIG. 20B. Through the insertion stage, the bone anchoring device 1101 may be coupled to tip 1180 but not threaded thereto and the tension of suture wires 1154 hold it in place. Coupling of the anchoring device 1101 with the delivery tool 1170 allows the suture wires 1154 of the anchoring device 1101 to extend through the lumen of the delivery tool shaft 1172 and the handle 1176 of the delivery system, and exit delivery tool 1170 through a suture wire opening 1157. The delivery tool 1170 further includes a delivery tool rotation mechanism 1182 which is coupled to an internally held delivery tool slider 1184 and which controls the deforming force applied by the delivery tool 1170 on the anchoring device as shown in FIG. 20C. The slider 1184 may be tubular. The slider 1184 may include at a proximal end a slider cap 1178. The suture wires 1154 may pass through the shaft 1172, optionally through slider 1184. The suture wires 1154 may be accommodated within the slider 1184. The rotation mechanism 1182 may be suitably coupled to the slider 1184. One non-limiting example of a suitable rotation mechanism 1182 may be an adjustment knob. The knob 1182 can be turned in one direction to pull the coupled slider 1184 and the wires 1154 longitudinally away from the tip 1180 of the delivery tool 1170 and towards the knob 1182. FIG. 20C shows the movement of the slider 1184 backwards with tightening of the suture wires 1154. The pulling may apply a deforming force on at least part of the anchoring device 1101. The applied force may facilitate the wings 1166 of the anchoring device 1101 to protrude out of the spaces 1164 of the tubular body 1161 of the anchoring device 1101 and radial expansion of the mesh 1122 as shown in FIG. 20C. The delivery tool 1170 may include a delivery tool stabilizing member 1188 for preventing unwanted rotation of the slider 1184, i.e., the slider 1184 may be designed to allow only axial movement. The stabilizing member 1188 is shown in an initial position of the slider 1184 in FIGS. 20A and 20B. The stabilizing member 1188 is shown in a subsequent position when the slider 1184 is pulled backward and the anchoring device 1101 is deployed in FIG. 20C. The stabilizing member 1188 may comprise a pin and constitutes an indication of the deployment of the bone anchoring device 1101.

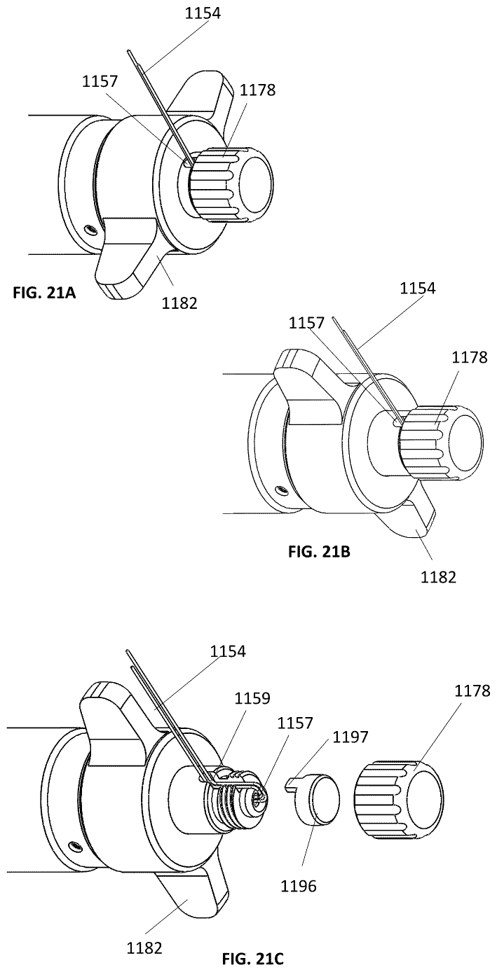

FIG. 21A is an isometric view of an enlarged proximal section of the delivery device 1170 in the initial state. FIG. 21B is an isometric view of an enlarged proximal section of the delivery device 1170 after rotation of the rotation mechanism 1182. FIG. 21C is an isometric view of an enlarged proximal section of the delivery tool 1170 after removing the wire cap 1196 and the slider cap 1178.

In some embodiments, to facilitate deployment of the bone anchoring device 1101, knob 1182 is rotated, optionally, counterclockwise and consequently the slider 1184 is moved proximally to facilitate deforming force on the anchoring device 1101 (FIG. 21B). In some embodiments, to facilitate detachment of the delivery tool 1170 from the anchoring device 1101, the wire cap 1196 and the slider cap 1178 may be removed from the delivery device 1170 as shown in FIG. 21C. The suture wires 1154 extend out from the delivery tool 1170 through wire opening 1157, and run along wire channel 1159. The wire cap 1196 is configured to protect the wire 1154 and to hold the wires 1154 tightly in the device via tab 1197. The wire cap 1196 may be made from a material, such as, but not limited to silicone. The slider cap 1178 may be configured to hold the slider 1184 in place. As such, before the delivery device is detached, these securing components 1178, and 1196 are removed. After delivery of the bone anchoring device 1101 into the bone and radial expansion of the mesh 1122, the anchoring device is suitably anchored in the bone. The delivery tool 1170 may be detached from the anchoring device 1101. The detachment may be by any suitable mechanism, including, but not limited to pulling out, and/or rotating movements.

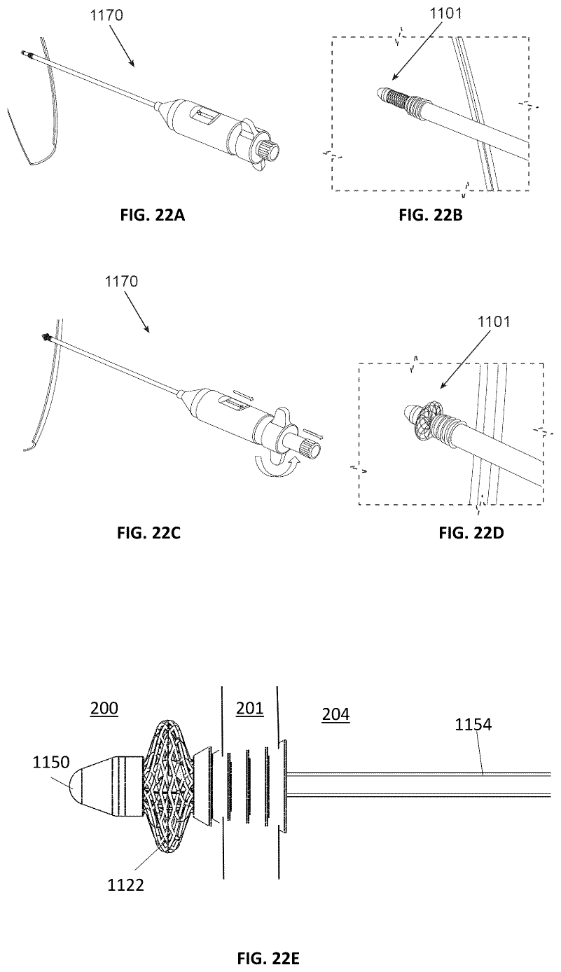

The surgical process, i.e., method of implementing the bone anchoring device 1101 described with reference to FIGS. 22A-22D, includes one or more of the following steps: (a) drilling into the bone to form a lumen, which is dimensioned to receive the bone anchoring device 1101; (b) inserting the bone anchoring device 1101 into the lumen within the bone using the delivery tool 1170 (FIGS. 22A-22B); the anchoring device 1101 is typically pushed inside the bone strongly enough to locate the anchor body 1124 of the implant into the cortical bone; (c) optionally screwing (i.e., by rotations) the bone anchor body 1124 into the bone; d) employing the delivery tool 1170 to radially expand the deformable structure 1160 and mesh 1122 by rotation of the rotation mechanism 1182 (FIGS. 22C-22D); (e) detaching the delivery tool 1170 from the implanted anchoring device 1101 by removing the cap 1196 and the slider cap 1178; (f) withdrawing the delivery tool; (g) applying suture wires 1155 and/or wires 1154 to the damaged tissue; and optionally (h) repeating the method for implanting any suitable number of anchoring devices 1101. The delivery tool 1170 may be for one time use and may be disposable. In some embodiments, each anchoring device 1101 is inserted using a new delivery tool 1170.

The delivered and implanted bone anchoring device 1101 may be located in the bone as shown in FIG. 22E such that at least part of the bone anchor body 1124 may be disposed in the cortex 201 of the bone. The tip 1150 of the anchoring device 1101 and the radially expanded mesh 1122 connected to the bone anchor body 1124 may be positioned inside the bone 200 where a spongy or cancellous bone tissue resides. A section of suture wire 1154 may extend from within the bone to outside the bone or body 204. The suture wire 1154/1155 may then be suitably employed, such as but not limited to being tied by a surgeon/user.

The bone anchoring device 1101 may remain inside the patient for any suitable period of time.

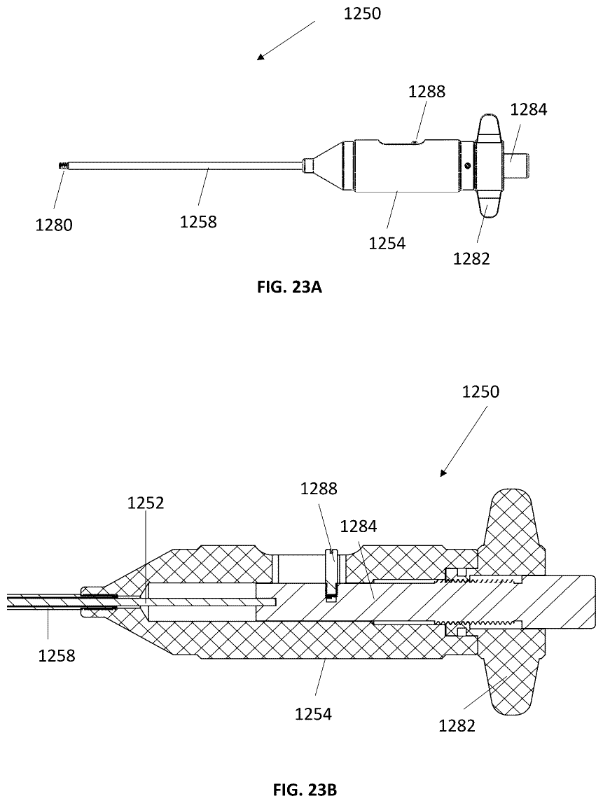

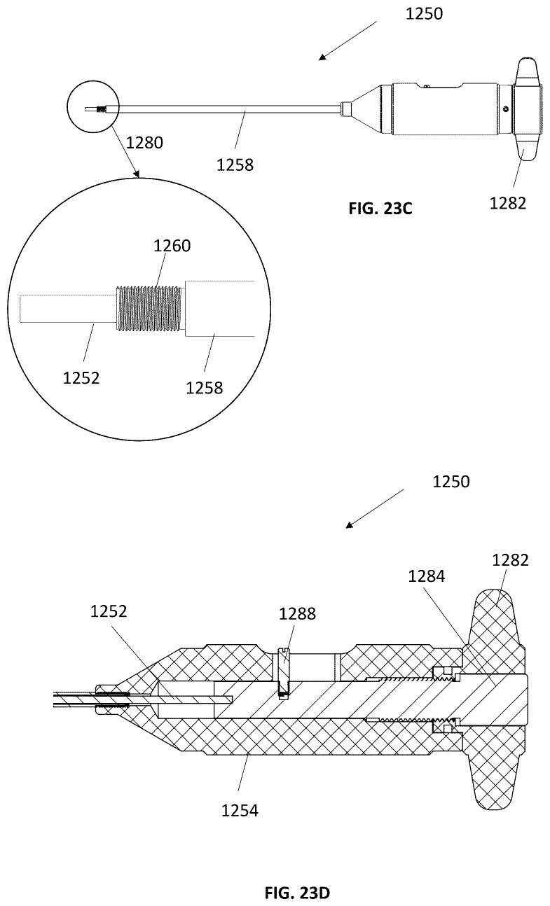

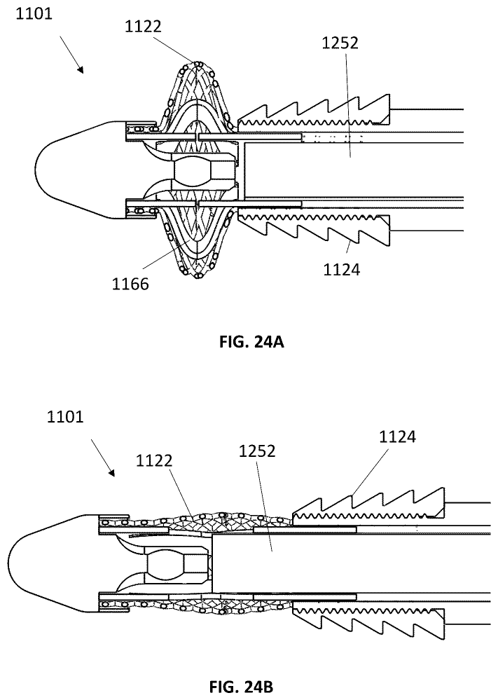

The bone anchoring device 1101 may be removed from the bone using a retrieving device 1250 of the present invention as shown in FIGS. 23-24. The retrieving device 1250 may be similar to the delivery tool 1170 described hereinabove for example in FIGS. 20-21. The retrieving tool 1250 may essentially include a retrieving tool shaft 1258, a handle 1254 and retrieving tool slider 1284. In contrast to the delivery tool 1170, the retrieving tool 1250 includes a retrieving slider 1284 configured to apply a pushing force on a pushing rod 1252 and thereby to apply a push force on at least part of the anchoring device 1101. The retrieving tool shaft 1258 may accommodate a pushing rod 1252 which may be coupled to a retrieving slider 1284 which is consequently coupled to the tool rotation mechanism 1282. The rotating mechanism 1282 may be a knob, which is turnable. The retrieving tool 1250 may include a retrieving tool stabilizing member 1288 to force the movement of the slider 1284 to be axial, i.e., prevent unwanted rotation of the slider 1284. The retrieving tool pushing rod 1252 may be coupled to the slider 1284 and may be moved when the slider 1284 is moved, which may be dictated by positioning of the rotation mechanism 1282, such as by turning the knob. The retrieving tool shaft 1258 is sized for insertion into a bone. The pushing rod 1252 includes at a distal end thereof a screw type thread 1260 (FIG. 23C).

The retrieving tool 1250 may have a plurality of configurations. FIGS. 23A-23B show a first configuration of the retrieving tool 1250. The first configuration may be adopted by the retrieving tool 1250 before extraction of the anchoring device 1101. In this configuration, the slider 1284 protrudes from the proximal end of the retrieving tool 1250 and has not been pushed distally to push the retrieving tool pushing rod 1252 along the device towards the retrieving tool tip 1280. The retrieving tool 1250 may be adaptable to a second configuration employed during retrieval of an anchoring device 1101. The second configuration may facilitate applying a pushing force on an attached anchoring device 1101 as shown in FIGS. 23C-23D. The knob 1282 is turned to a position which is configured to push the retrieving tool slider 1284 into the device to push the pushing rod 1252, so that it protrudes from the tip 1280 of the retrieving tool 1250 as shown in FIGS. 23C-23D.

The tip 1280 of the retrieving tool 1250 may couple with the implanted anchoring device 1101. The retrieving tool 1250 may be inserted into the surgical space and maneuvered to attach the anchoring device 1101 to the tip 1280 of the retrieving tool. In an initial attachment configuration of the retrieving tool 1250 coupled to the anchoring device 1101, the mesh 1122 of the anchoring device may be in a radially expanded conformation and the pushing rod 1252 may be positioned so that it does not provide a pushing force on the attached anchoring device as shown in FIG. 24A. The rotating knob mechanism 1282 of the retrieving tool 1250 may be switched to a position which axially pushes the slider 1284 and the pushing rod 1252 towards and through the retrieving tool tip 1280 and applies a pushing force on the distal end of the attached anchoring device 1101 as shown in FIG. 24B. The pushing force collapses the deformable structure 1160 of the bone anchoring device 1101 so that the wings 1166 flatten to return to lie within the spaces 1164 and in the plane of the deformable structure 1160. The pushing force from the pushing rod 1252 further pull the mesh 1122 longitudinally and reconfigure the radially expanded mesh to lie flat surrounding the deformable structure 1160. In its undistorted, straightened configuration the anchoring device 1101 may be removed from the bone and out of the body.

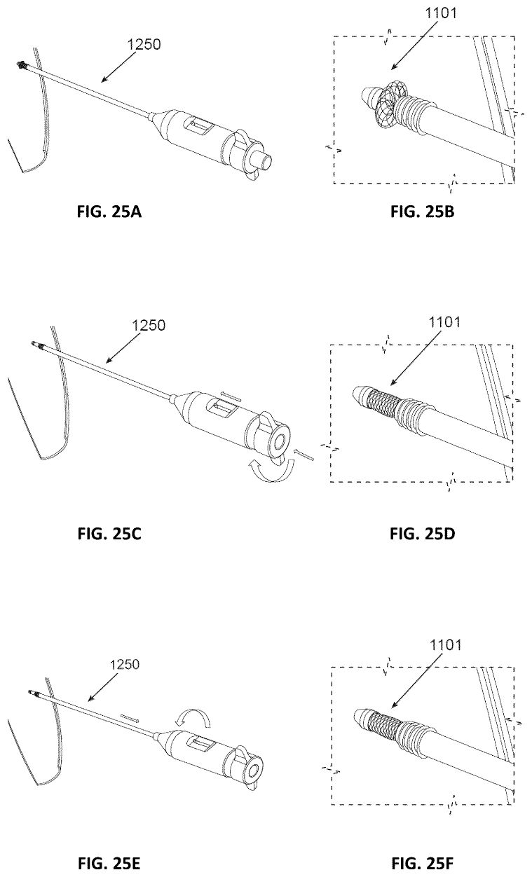

FIG. 25A is an isometric view illustrating the herein bone anchoring device 1101 when implanted within a bone and connected to a retrieving tool 1250. FIG. 25B is an enlarged section of the distal section of the retrieving tool 1250 when connected to the bone anchoring device 1101. The bone anchoring device 1101 is in an expanded state. FIG. 25C is an isometric view illustrating the herein bone anchoring device 1101 when in a collapsed state and connected to a retrieving tool 1250. FIG. 25D is an enlarged section of the distal section of the retrieving tool 1250 when connected to the bone anchoring device 1101. The bone anchoring device 1101 is collapsed as a result of the user that pushed the retrieving tool 1250 distally into the bone by rotating clockwise the rotation mechanism. FIG. 25E is an isometric view illustrating the herein bone anchoring device 1101 when in a collapsed state and connected to a retrieving tool 1250. FIG. 25F is an enlarged section of the distal section of the retrieving tool 1250 when connected to the bone anchoring device 1101. The bone anchoring device 1101 is in a collapsed state and the user can pull out the retrieving tool 1250 while pulling out and/or rotating counter-clockwise (right-hand opening action). The left-hand thread 1140 in the implant's anchoring part will remain tightly attached to the tool.

FIG. 2629 show yet another exemplary bone anchoring surgical system 1300 comprising a bone anchoring device 1301 in accordance with embodiments of the present invention that may be used for example in shoulder arthroscopy procedures, such as of the rotator cuff tears repair.

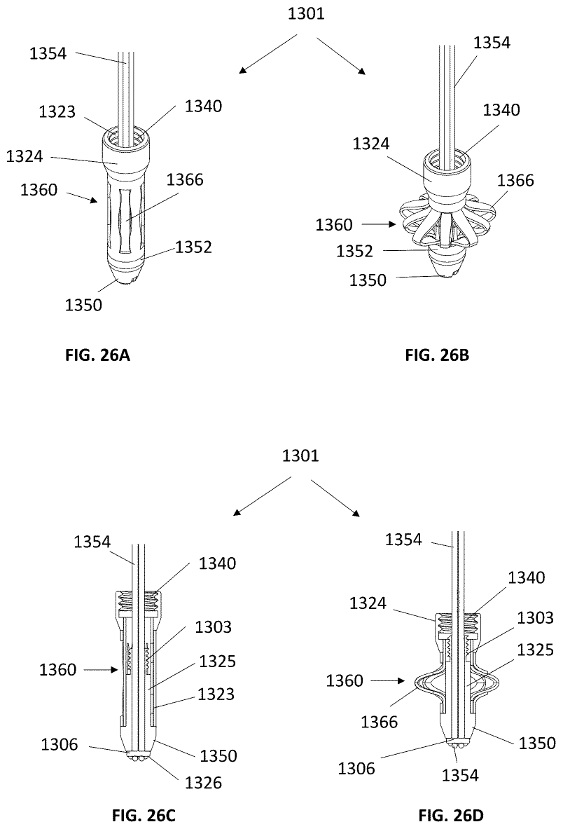

FIG. 26A is an isometric view of bone anchoring device 1301 when in a collapsed/non-expanded state. FIG. 26B is an isometric view of bone anchoring device 1301 when in an expanded state. FIG. 26C is a cross section view of bone anchoring device 1301 in the collapsed/non-expanded state. FIG. 26D is a cross section view of bone anchoring device 1301 in the expanded state.

From FIGS. 26A-26D it can be seen that bone anchoring device 1301 has a deformable structure 1360 that can radially expand as shown in FIGS. 26B and 26D. Anchoring device 1301 may or may not include a mesh (not shown) that wraps the entire surface of deformable structure 1360. The bone anchoring device 1301 may include a bone anchor body 1324, featured by an internal thread 1340 or a ribbed element for connecting to a delivery tool by threading. The bone anchor body 1324 and device 1301 may have a central longitudinal cavity 1323. The bone anchor body 1324 may be disposed at the proximal end of the device 1301. At the distal end of the device 1301 is a device tip 1350. The tip 1350 may have an opening in a form of an eyelet 1326 at its distal extremity. The tip 1350 may be rigid. The tip 1350 may be coupled to a support ring 1352, which may be positioned at the proximal base of the tip 1350. Anchoring device 1301 may further include one or more suture wires 1354. The suture wires 1354 may be accommodated in the cavity 1323 of the anchoring device 1301. The suture wires 1354 may be surgical wires, suitable to suture body tissues, such as an injured tissue (i.e., a torn tendon). The suture wires 1354 may be seen through eyelet 1326 disposed at the distal most end of tip 1350. Suture wires 1354 may be supported by a distal wire fixation element 1306. Suture wires 1354 may pass and extend along the entire length of anchoring device 1301.

The deformable structure 1360 may be coupled at a distal end to the tip 1350 of the device 1301, and at a proximal end to the bone anchor body 1324 such that the deformable structure 1360 is disposed and extend between the device tip 1350 and the bone anchor body 1324.

The deformable structure 1360 comprises a plurality of deformable wings or strips 1366. The deformable structure 1360 may be constructed from a material so that it is reversibly deformable and may be suitably deformed on application of a suitable force applied thereon. For example, when pushed proximally (i.e., toward the physician), the deformable structure 1360 may be deformed into an expanded or contracted state, as shown in FIGS. 26B and 26D and when a reverse force applied it may adopt a collapsed or non-contracted/non-expanded state (as shown in FIGS. 26A and 26C). The deformable structure 1360 may be made from a material such as, but not limited to titanium, nitinol and stainless steel.

Within its cavity 1323 bone anchoring device 1301 includes a central longitudinal column 1325 that connects the bone anchor body 1324 to the tip 1350. Anchoring device 1301 may further include a proximal column thread 1303 on column 1325. Column thread 1303 is configured to allow coupling to a moving part of the delivery tool (i.e., delivery tool slider 1484 that is moving proximally toward the physician).

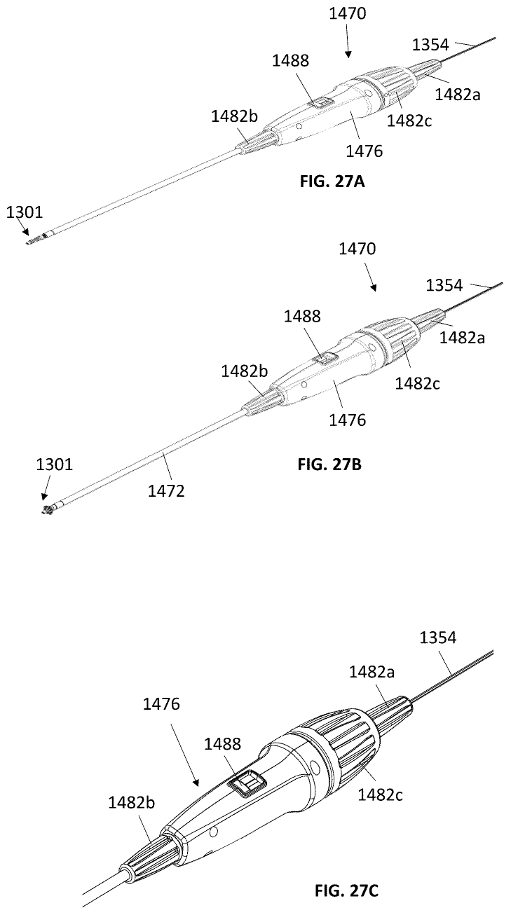

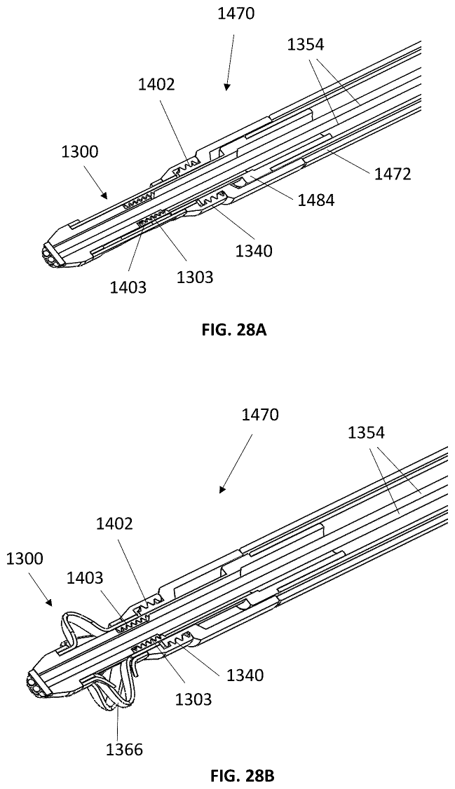

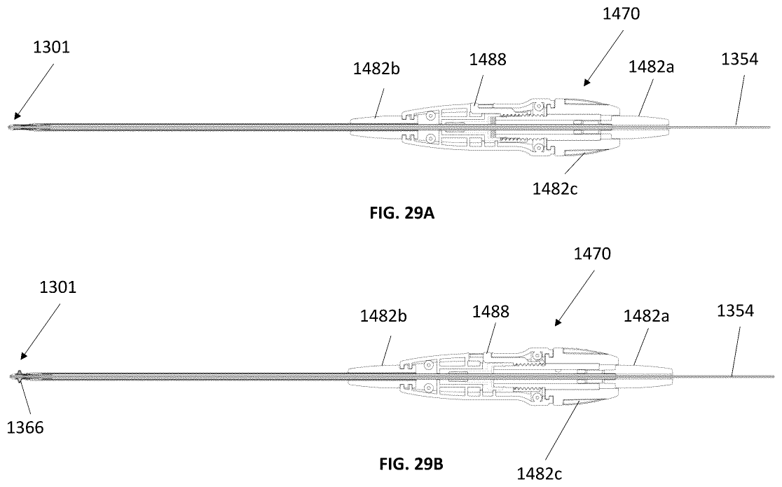

FIGS. 27A-27C and 28A-28B show an exemplary delivery tool 1470 which may be used to insert a bone anchoring device 1301 to a bone in accordance with embodiments of the present invention. The delivery tool 1470 includes a longitudinal delivery tool shaft 1472, a delivery tool handle 1476, and an internal delivery tool slider 1484 comprising a distal slider thread 1403. The delivery tool 1470 comprises a first proximal rotating knob 1482a that controls the connection between internal column thread 1303 and slider thread 1403. The delivery tool 1470 comprises a second distal rotating knob 1482b that connects another second delivery tool thread 1402 (shaft thread) to the bone anchor body internal thread 1340. A central rotating knob 1482c moves the distal part of the anchoring device 1301 proximally and provides opening of the anchor wings or strips 1366. The expansion of the anchor wings or strips 1366 can be viewed by an indicator 1488.

FIG. 27C illustrates handle 1476 of delivery tool 1470. Handle 1476 comprises a first distal rotating knob 1482a, a second proximal rotating knob 1482b, and a third central rotating knob 1482c. Suture wires 1354 extend from distal tip 1350 of bone anchoring device 1301 through the internal lumen of delivery tool 1470 and to the exterior of devices 1301 and 1470. Handle 1476 further includes an indicator 1488 that indicates the state of anchoring device, i.e., whether it is in the collapsed or non-contracted/non-expanded state as shown in FIG. 29A or the expanded or contracted state as shown in FIG. 29B.

FIGS. 29A-29B show bone anchoring device 1301 when connected to delivery tool 1470. FIG. 29A shows bone anchoring device 1301 when in the collapsed or non-contracted/non-expanded state. FIG. 29B shows the expanded or contracted state of bone anchoring device 1301. To facilitate expansion or contraction of wings 1366, rotation of central rotating knob 1482c of the delivery tool 1470 should be conducted to thereby move the distal part of the anchoring device 1301 proximally toward a physician. Such rotation of central rotating knob 1482c moves slider 1484 and indicator 1488 proximally and provides opening of the anchor wings 1366. The delivery tool 1470 can be disconnected from anchoring device 1301 by rotating knob 1482a to thereby release internal column thread 1303 from slider thread 1403 (shown in FIGS. 28A and 28B) and then rotating second rotating knob 1482b to thereby release shaft thread 1402 from the bone anchor body internal thread 1340 (shown in FIGS. 28A and 28B).

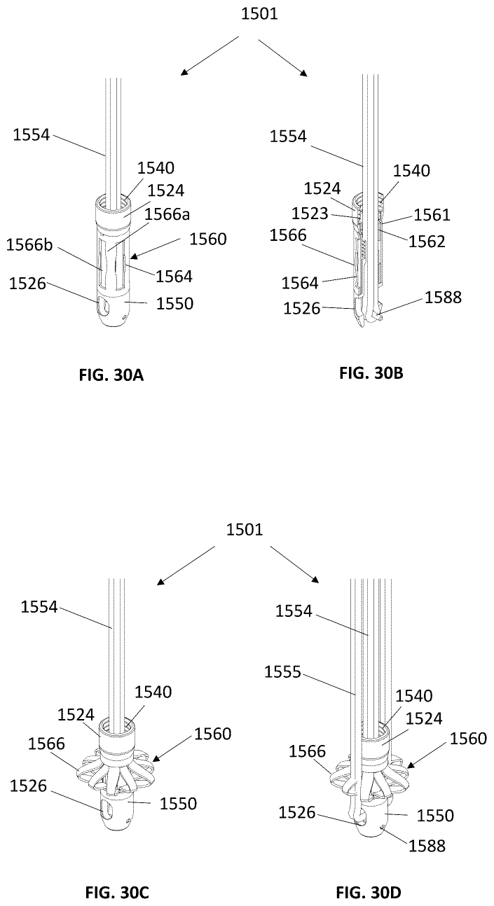

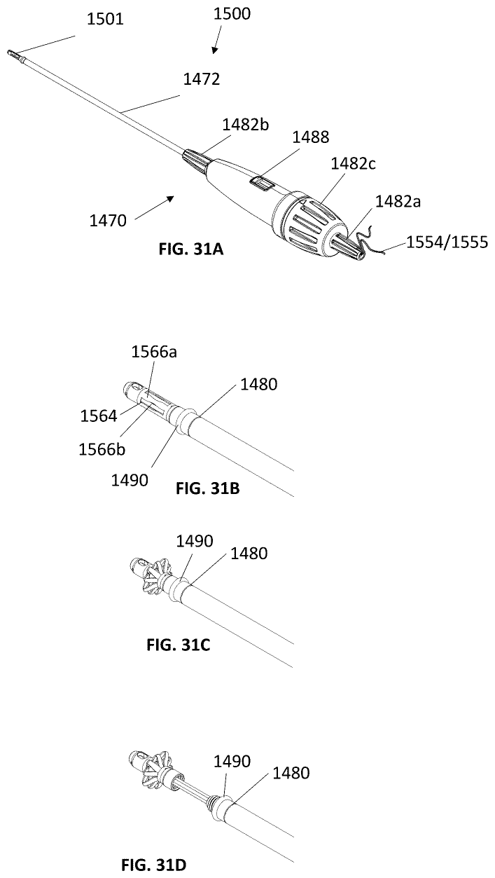

FIGS. 30A-30D illustrate yet another exemplary bone anchoring device 1501 according to an aspect of the present invention. Bone anchoring device 1501 comprises a proximal bone anchor body 1524, a distal tip 1550, and deformable structure 1560 disposed between the proximal bone anchor body 1524 and the distal tip 1550. Deformable structure 1560 is movable between a collapsed configuration as shown in FIGS. 30A and 30B and an expanded configuration as shown in FIGS. 30C and 30D. Bone anchoring device 1501 may or may not include a mesh (not shown) that wraps the entire outer surface of deformable structure 1560. Bone anchoring device 1501 may include a proximal bone anchor body 1524 comprising an internal thread 1540 configured for connecting to a delivery tool, for example by threading i.e., to delivery tool shaft 1472 (FIG. 31A). The outer surface of bone anchor body 1524 and/or of distal tip 1550 may be smooth. At the distal end of device 1501 there is device tip 1550. In some embodiments, the bone anchor body 1524 may have an outer diameter that may be larger than the outer diameter of tip 1550 to secure the implantation within the bone. In an alternative embodiment, the outer diameter of both the distal tip 1550, and the bone anchor body 1524 are similar and may be similar to the outer diameter of deformable structure 1560 when in a non-expanded state. Tip 1550 may comprise a front opening at its distal extremity or may be closed.

Distal tip 1550 may comprise one or more suture wire couplers, such as eyelet opening 1526 that forms a transverse channel in the body of distal tip 1550. Various suture wire coupling members are contemplated and applicable. Exemplary suture wire couplers include an eyelet, a pin, an anchor or the like. Eyelet 1526 allows passing therethrough one or more suture wires 1555 that can be used to suture an injured tissue, such a torn tendon. Eyelet 1526 allows passing therethrough one or more than one (e.g., two or three, or four) suture wires 1555. Suture wire 1555 can be attached to the bone anchoring device 1501 or slide about eyelet 1526. Suture wire 1555 may pass outside of device 1501 and extend along the entire outer length of bone anchoring device 1501 and inside delivery tool 1470.

Device 1501 may include a second suture wire coupler comprising pin 1588. Pin 1588 extends internally in distal tip 1550 and is configured to allow accommodating suture wire 1554 in cavity 1523 of device 1501. Suture wire 1554 may be wrapped around pin 1588. Suture wire 1554 can be attached to the bone anchoring device 1501 or slide inside the cavity 1523 of the device 1501 around internal distal pin 1588. Suture wire 1554 may pass and extend along the entire length of anchoring device 1501 and delivery tool 1470. Pin 1588 can couple one or more than one (e.g., two or three, or four) suture wires 1554.

The deformable structure 1560 comprises a plurality of deformable wings 1566. The deformable structure 1560 may be constructed from a material so that it is reversibly deformable and may be suitably deformed on application of a suitable force applied thereon. For example, when deformable structure 1560 is pushed proximally (i.e., toward the physician), the deformable structure 1560 may be deformed into an expanded or contracted state, as shown in FIGS. 30C and 30D. When a reverse force applied it may adopt a collapsed or non-contracted/non-expanded state (as shown in FIGS. 30A and 30B).

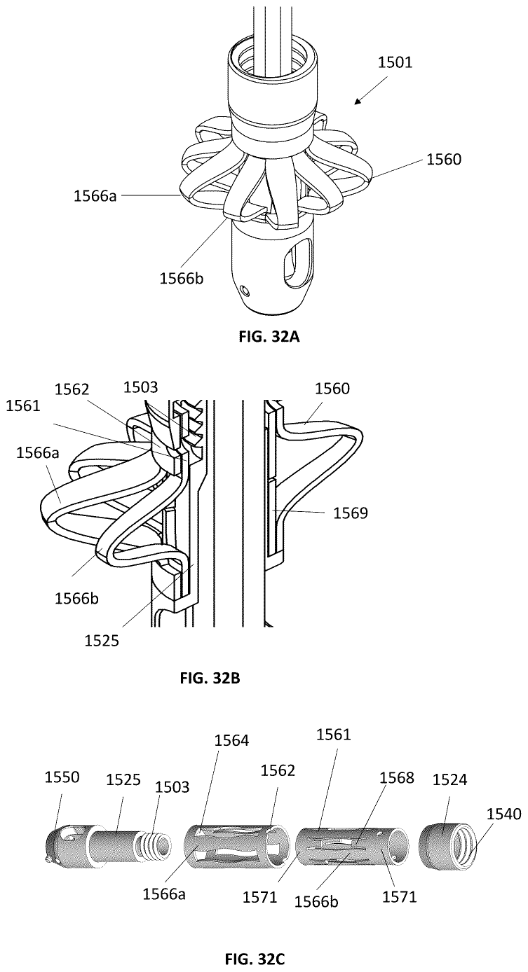

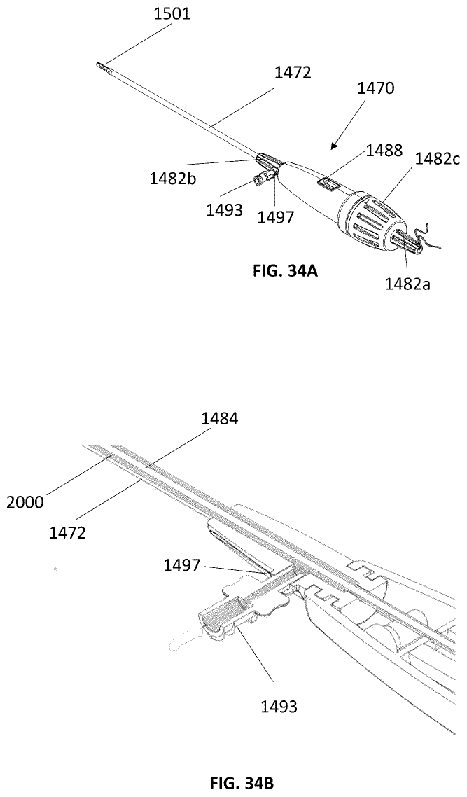

FIGS. 31A-31D show delivery tool 1470 that comprises a shaft 1472, and a handle 1476 comprising three rotation mechanisms. Various rotation mechanisms are herein contemplated including without limitation a knob, ball bearing, roller and the like. The rotation mechanisms include a proximal first rotating knob 1482a, a second distal rotating knob 1482b, and a third central rotating knob 1482c. The delivery tool 1470 comprises an elongated shaft 1472 that can be coupled to the bone anchoring device 1501 and accommodate suture wires 1554, and/or 1555 that are connected to device 1501 and pass through the delivery tool 1470. Delivery tool 1470 accommodates an internal slider (such as slider 1484 of FIG. 34B) that extends between the distal and proximal extremities of delivery tool 1470. Delivery tool 1470 is connected to device 1501. Shaft 1472 is connected to internal thread 1540 of bone anchor body 1524. Slider 1484 is connected to column thread 1503 on central column 1525 (shown in FIGS. 32B-32C).

By rotating (e.g., clockwise) the central rotating knob 1482c, delivery tool 1470 transforms bone anchoring device 1501, from a collapsed state (FIG. 31B) to an expanded state (FIG. 31C). By rotating (e.g., counterclockwise) the proximal rotating knob 1482a, delivery tool slider 1484 is detached from device 1501. By (e.g., counterclockwise) rotating knob distal 1482b, shaft 1472 is detached from device 1501 moving it to a delivery state (FIG. 31D) where the bone anchoring device 1501 is released from the delivery tool 1470. The delivery tool 1470 may also have one or more distal markers 1480 and a protruding protection ring 1490 to protect from inserting the bone anchoring device 1501 too deep into the bone.

FIG. 32A shows bone anchoring device 1501 which comprises two layers of wings 1566, outer wings 1566a and inner wings 1566b. In one non-limiting example, the deformable structure 1560 may comprise a first and a second overlapping tubular bodies 1561 and 1562. Each of first and second overlapping tubular bodies 1561 and 1562 comprises wings 1566. First and second overlapping tubular bodies 1561 and 1562, respectively, may comprise a plurality of wings 1566. For example, the deformable structure 1560 can comprise one, two, three, four, five, six, seven, eight or more outer wings 1566a. Each possibility represents a separate embodiment of the invention. The deformable structure 1560 may comprise one, two, three, four, five, six, seven, eight or more inner wings 1566b. Each possibility represents a separate embodiment of the invention. In an exemplary embodiment, there are four outer wings 1566a and four inner wings 1566b.

In one non-limiting example, the deformable structure 1560 may comprise a first and a second overlapping tubular bodies 1561 and 1562, each comprising wings 1566 (shown for example in FIGS. 30B and 32C). A first outer tubular body 1561 may comprise a plurality of spaces (e.g., laser-cuts) 1564 configured to accommodate deformable wings 1566b when the deformable structure 1560 is in an expanded state. Such configuration provides a structure wherein outer 1566a and inner 1566b wings may be arranged alternately, with an outer wing 1566a next to an inner wing 1566b. The two types of wings protrude outwardly. The outer wings 1566a may, but not necessarily extend deeper into the bone cortex than the inner wings 1566b. This structure of deformable structure 1560 enhances stability, expands the fixation and contact surface with the bone, increases pull-out force resistance, and significantly improves the osteointegration potential between the bone and the device 1501. FIG. 32B shows a magnified cross section of a portion of the bone anchoring device 1501, and FIG. 32C is an exploded view of bone anchoring device 1501. The figures show the outer wings 1566a and inner wings 1566b. The deformable structure 1560 comprises two overlapping tubular bodies, each comprising wings 1566 wherein an outer tubular body 1562 comprises the outer wings 1566a and the inner tubular body 1561 comprises the inner wings 1566b and further comprises stoppers 1569 disposed between the inner wings 1566b. Outer tubular body 1562 may comprise wings 1566a and cutouts/spaces 1564 therebetween. Inner tubular body 1561 may comprise wings 1566b and ribs 1568 therebetween. Extending from distal tip 1550 and disposed in cavity 1523 is a central longitudinal column 1525. At the proximal end of column 1525 there is column thread 1503 configured to couple cooperating thread (not shown) on slider 1484 of delivery tool 1470.

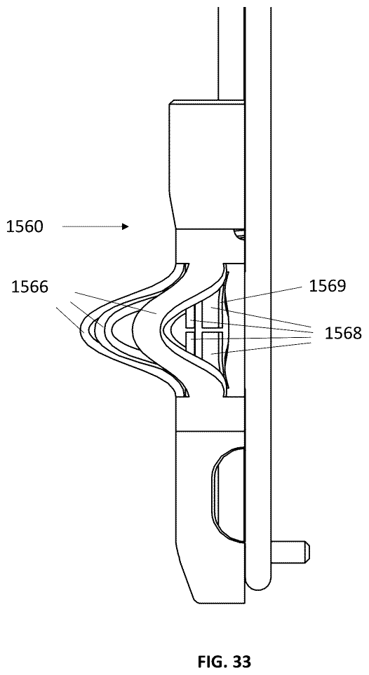

FIG. 33 illustrates bone anchoring device 1501 which further comprises at least one stopper 1569 that provides support to the wings 1566 and restricts the extent of wings 1566 contraction. Optionally, the stoppers 1569 afford maintaining a space or angle of above about 10 degrees between the distal and proximal parts of the wings 1566. For example, the stoppers 1569 afford maintaining an angle of above about 15, or above about 20 degrees between the distal and proximal parts of the wings 1566. The at least one stoppers 1569 include structural stabilizing ribs 1568 that prevent the anchoring device 1501 from being overly deformed. The deformable structure 1560 may feature at least one stopper 1569. The deformable structure 1560 may include two or more, three or more, four or more, five or more, six or more, or seven or more stoppers 1569 disposed between wings 1566b. Each possibility represents a separate embodiment of the invention. For example, the deformable structure 1560 comprises four stoppers. For example, the deformable structure 1560 comprises five stoppers 1569. Each stopper 1569 may comprise two opposing ribs 1568 extending from the two base elements 1571 of tubular body 1561 of deformable structure 1560. The ribs 1568 may be rigid and may limit the deformable structure 1560 to contract up to a length which is equivalent to twice or more the length of two ribs 1568.

FIGS. 34A-34B shows delivery tool 1470 which may further comprise a port 1497 for connecting an injection tube 1493 configured to introduce a filling material 2000 into a bone anchoring device 1501 in accordance with embodiments of the present invention. The filling material 2000 may be introduced through port 1497 attached to knob 1482a or any other portion of delivery tool 1470. The filling material 2000 may be injected into the delivery tool shaft 1472, between the shaft 1472 and the slider 1484.

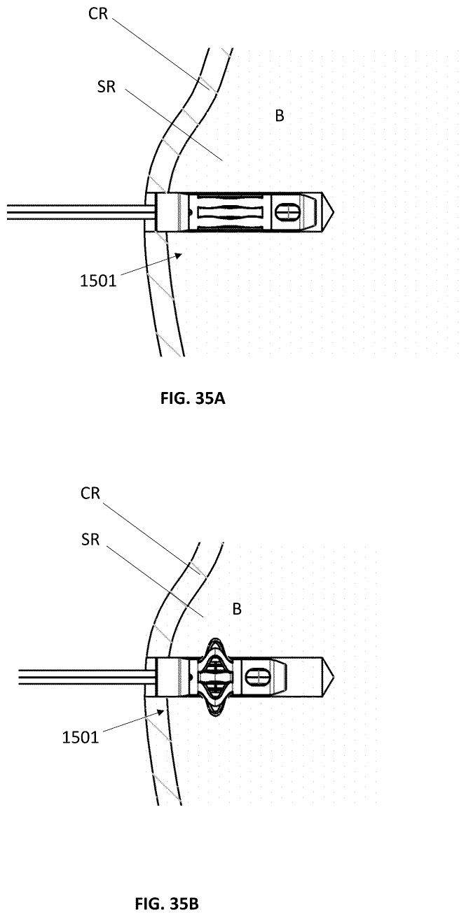

FIGS. 35A-35B show a bone anchoring device 1501 when placed inside bone B, having a cortical region CR and a sponge bone region SR. The bone anchoring device 1501 is shown in a contracted/non-expanded state (FIG. 35A) and in an expandable state (FIG. 35B).

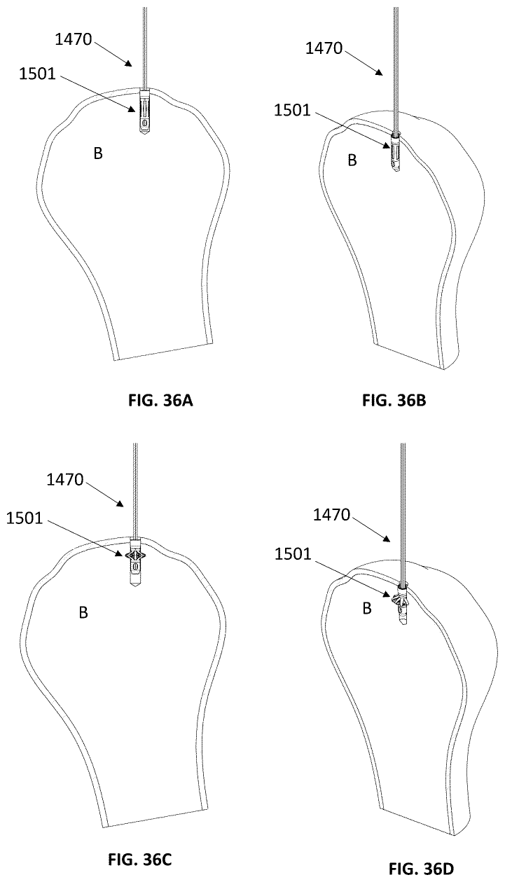

FIGS. 36A-36D show a bone anchoring device 1501 attached to a delivery tool 1470 when placed inside a humerus for repairing a torn rotator cuff.

An exemplary method of using the device 1501 includes, for example, (a) drilling into the humerus bone to form a lumen dimensioned to receive device 1501; (b) inserting tip 1550 of device 1501 into the lumen; (c) inserting at least the distal tip 1550 into the lumen of the humerus bone, for example, by striking anchor body 1524 using a hammer until the device 1501 is fully within the bone; (d) turning central deployment knob 1482c, optionally in a clockwise direction, optionally until reaching a full stop wherein indicator 1488 is fully moved proximally to thereby indicate of full deployment; (e) turning the proximal release knob 1482a, optionally, counterclockwise until slider 1484 disengages from device 1501; (f) turning the distal release knob 1482b, optionally, counterclockwise until shaft 1472 disengages from device 1501; (g) pulling the delivery tool 1470 out from the bone and the body of the subject to thereby reveal the suture wires 1554/1555; and (h) using the suture wires 1554/1555 for suturing a ligament, or tendon such as the rotator cuff.

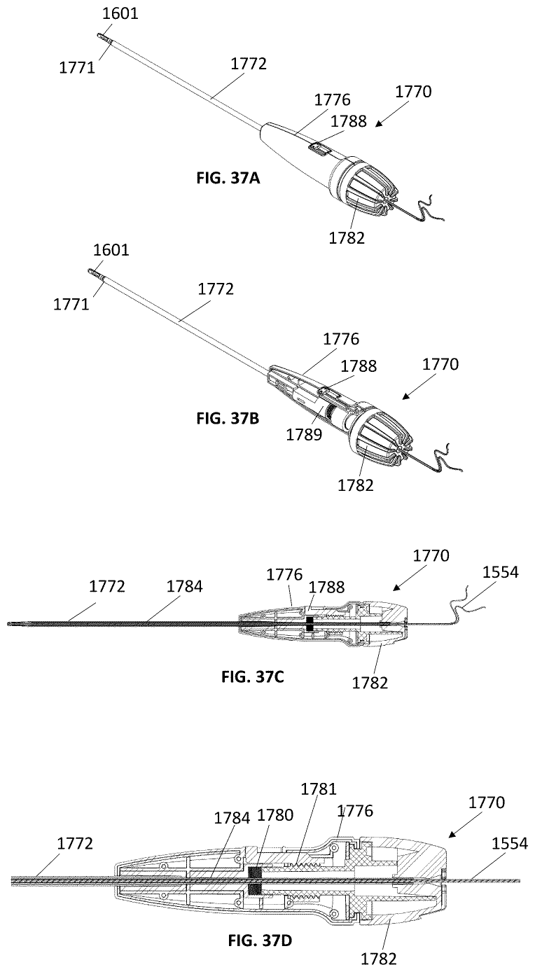

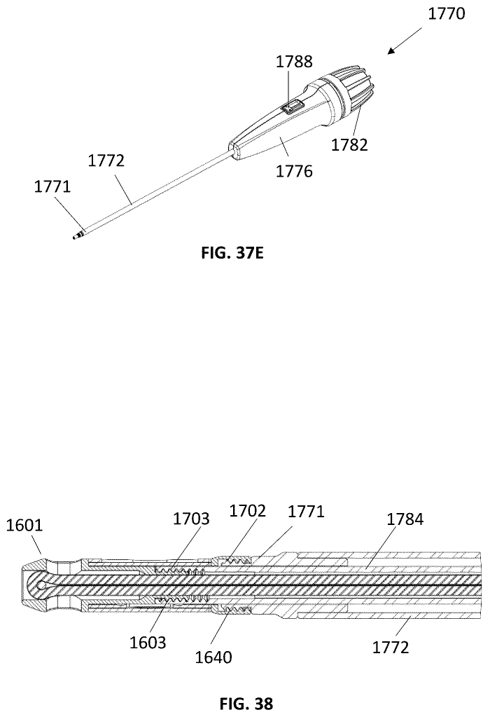

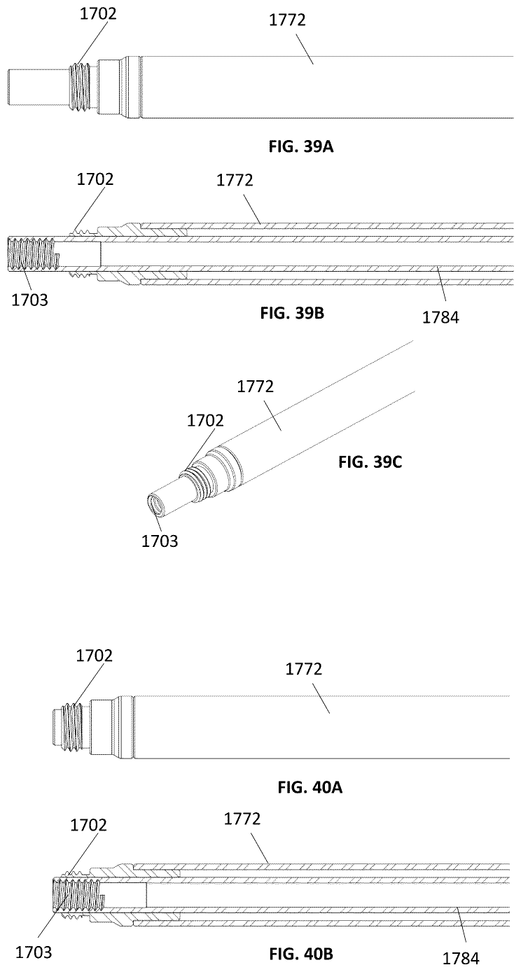

FIGS. 37A-37E show yet another exemplary delivery tool 1770 which may be used to insert and deploy a bone anchoring device as herein disclosed in accordance with embodiments of the present invention. Delivery tool 1770 may be configured to insert and deploy various bone anchoring devices, such as the devices disclosed herein, including devices 1101, 1301, and 1501, and 1601. The delivery tool 1770 includes an elongated delivery tool shaft 1772 that can be coupled to the bone anchoring device, such as bone anchoring device 1601 (shown in FIGS. 39A-39D). The delivery tool 1770 is configured to accommodate suture wires, such as suture wires 1554, and/or 1555 that are connected to device 1601 and pass through the delivery tool 1770.

The delivery shaft 1772 may be elongated and extend distally from the delivery handle 1776.

The delivery tool 1770 may include a delivery slider 1784 disposed within and extending longitudinally through the delivery shaft 1772. Slider 1784 may extend between the distal and proximal ends of delivery tool 1770. Slider 1784 may be configured to move axially within the delivery shaft 1772 and tool 1770. Delivery tool device 1770 further includes a delivery tool handle 1776 operatively connected to the delivery slider 1784. A rotation mechanism, such as rotation knob 1782 may be configured to control axial movement of the delivery slider 1784 when rotated.

Internal delivery tool slider 1784 extends along the length of delivery tool 1770, i.e., from distal tip 1771 of delivery tool 1770 via shaft 1772 and out of delivery tool 1770 through handle 1776. Within slider 1784 which constitutes a hollowed tube, resides suture wire 1554/5.

As shown in FIG. 38, delivery tool 1770 is connected to device 1601. Delivery tool 1770 includes at its distal tip 1771 a shaft thread 1702 on its exterior surface. The shaft thread 1702 may be configured to engage with a first thread (e.g., internal thread) 1640 of the bone anchoring device 1601. The delivery slider 1784 may include a slider thread 1703 configured to engage with a second thread (e.g., column thread) 1603 of the bone anchoring device 1601. This threaded interface between the delivery slider 1784 and the bone anchoring device may allow controlled deployment of the bone anchoring device through manipulation of the delivery tool components.

As illustrated, the delivery tool 1770 may comprise only one rotation mechanism as rotating knob 1782 that controls the connection between column thread 1603 of bone anchoring device 1601 and slider thread 1703 and maneuvers the expansion of the wings of anchoring device 1601. Various rotation mechanisms are herein contemplated including without limitation a knob, ball bearing, roller and the like. Rotating knob 1782 moves anchoring device 1601 proximally and provides opening of the anchor wings or strips (not shown). The expansion of the anchor wings or strips can be viewed by an indicator 1788 that provides visual feedback on the state of bone anchoring device, i.e., whether it is in the collapsed or non-contracted/non-expanded state.

To facilitate expansion or contraction of wings, rotation of rotating knob 1782 of the delivery tool 1770 should be conducted to thereby move anchoring device 1601 proximally toward a physician. Such rotation of rotating knob 1782 moves slider 1784 and indicator 1788 proximally and provides opening of the anchor wings.

As shown in FIGS. 37B and 37D, slider 1784 is coupled to knob 1782 via internal rotating member 1789. The rotating member 1789 may be configured to convert rotational motion of the rotation knob 1782 into linear motions of the delivery slider 1784 and indicator 1788. The deployment indicator 1788 may move in conjunction with the delivery slider 1784 as the rotation knob 1782 is actuated. Fine thread 1780 facilitates movement of slider 1784 whereas coarse thread 1781 of internal rotating member 1789 facilitates movement of indicator 1788.

The coarse thread 1781 may be designed to interact with the deployment indicator 1788. The larger pitch of the coarse thread 1781 may allow for more rapid movement of the deployment indicator 1788 relative to the movement of the delivery slider 1784. This configuration may provide enhanced visual feedback to the user regarding the deployment status of the bone anchoring device, as small rotations of the rotation knob 1782 may result in more noticeable movements of the deployment indicator 1788.

The combination of fine thread 1780 and coarse thread 1781 on the rotating member 1789 may allow for both precise control of the bone anchoring device deployment and clear visual indication of the deployment progress. This dual-threaded system may enhance the overall usability and effectiveness of the delivery tool 1770 during surgical procedures.

The delivery tool 1770 can be disengaged from anchoring device 1601 by rotating delivery tool 1770, or a portion thereof, such as handle 1776, and/or shaft 1772, to thereby release the connection between internal column thread 1603 of anchoring device 1601 from slider thread 1703 and further release shaft thread 1702 from the bone anchor body internal thread 1640 (shown in FIG. 38).

By rotating (e.g., clockwise) the rotating knob 1782, delivery tool 1770, transforms bone anchoring device 1601, from a collapsed state to an expanded state. By rotating (e.g., counterclockwise) the delivery tool 1770 (e.g., shaft 1772 and/or handle 1776), both delivery tool slider 1784 and shaft 1772 are detached from device 1601 and the bone anchoring device 1601 is released from the delivery tool 1770.

Optionally, the shaft thread 1702 and the slider thread 1703 may have essentially similar thread pitches. Similarly, the first thread 1640 and the second thread 1603 of the bone anchoring device may have similar pitches. This similarity in thread pitch may allow for simultaneous disengagement of both threaded interfaces from the bone anchoring device. As a result, the delivery tool 1770 may be detached from the bone anchoring device in a single step, which may simplify the surgical procedure and reduce the time required for device deployment. The coordinated disengagement of these threads may provide a smooth and efficient transition from the deployment phase to the release of the bone anchoring device within the surgical site.

Optionally, the delivery tool 1770 may be configured to allow detachment from the bone anchoring device through a swiveling motion. This swiveling action may involve rotating the delivery device or a portion thereof, such as the delivery handle 1776 or the shaft 1772. By performing this swiveling motion, both the delivery slider 1784 and the delivery shaft 1772 may be simultaneously released from their respective engagements with the bone anchoring device.

The swiveling motion may cause the shaft thread 1702 on the tool tip 1771 to disengage from the internal thread 1640 of the bone anchoring device, while concurrently causing the slider thread 1703 on the delivery slider 1784 to disengage from the column thread 1603 of the bone anchoring device. This coordinated disengagement may be facilitated by the similar thread pitches of these components.

The ability to detach the delivery tool 1770 from the bone anchoring device in a single step through a swiveling motion may offer several advantages. It may simplify the surgical procedure, potentially reducing the time required for device deployment and minimizing the risk of complications associated with multiple detachment steps. Additionally, this single-step release mechanism may provide the surgeon with improved control and efficiency during the final stages of the bone anchoring device implantation.

An exemplary method of using a device, for example device 1601 includes, for example, (a) drilling into the humerus bone to form a lumen dimensioned to receive device 1601; (b) inserting a tip of device 1601 into the lumen; (c) inserting at least the distal tip into the lumen of the humerus bone, for example, by striking the delivery tool 1770 when attached to device 1601 using a hammer until the device 1601 is fully within the bone; (d) turning deployment knob 1782, optionally in a clockwise direction, optionally until reaching a full stop wherein indicator 1788 is fully moved proximally to thereby indicate of full deployment; (e) turning delivery tool 1770, optionally, counterclockwise until slider 1784 and shaft 1772 disengage from device 1601; (g) pulling the delivery tool 1770 out from the bone and the body of the subject to thereby reveal the suture wires 1554/1555; and (h) using the suture wires 1554/1555 for suturing a ligament, or tendon such as the rotator cuff.

As used herein the term “distal” refers to the part of the device (either the implantable device or delivery/retrieving tool) which following implantation, is located farther into the body of the patient.

As used herein the term “proximal” refers to the part of the device (either the implantable device or delivery/retrieving tool) which is located closest to the point of entry of a patient/subject.

Each of the following terms: ‘includes’, ‘including’, ‘has’, ‘having’, ‘comprises’, and ‘comprising’, and their linguistic equivalents, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof.

The term ‘consisting essentially of” as used herein means that the scope of the claim is limited to the specified elements and those that do not materially affect the basic and novel characteristic(s) of the claimed device and materials.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

All ranges disclosed herein include the endpoints. The use of the term “or” shall be construed to mean “and/or” unless the specific context indicates otherwise.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the broad scope of the appended claims.

All publications, patents, and patent applications mentioned in this specification are herein incorporated in their entirety by reference into the specification, to the same extent as if each individual publication, patent, or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

It should be understood that the above description is merely exemplary and various embodiments of the present invention may be devised, mutatis mutandis, and that the features described in the above-described embodiments, and those not described herein, may be used separately or in any suitable combination; and the invention can be devised in accordance with embodiments not necessarily described above.

1. A bone anchoring surgical system comprising
a bone anchoring device configured to anchor a bone, comprising
a proximal bone anchor body;
a distal tip; and
a deformable structure disposed between the proximal bone anchor body and the distal tip, wherein the deformable structure comprises a plurality of deformable wings configured to move between a non-expanded configuration and an expanded configuration at a target skeletal position, wherein in the non-expanded configuration, the deformable wings are held flat in the deformable structure and wherein in the expanded configuration, the deformable wings are extended out of the deformable structure and protrude radially outward from the bone anchoring device.

2. The device of claim 1, further comprising an expandable mesh that encircles said deformable structure.

3. The system of claim 1, wherein the deformable structure is configured to be filled with a filler material selected from the group consisting of: bone cement; autologous bone; a bone substitute; a polymer; a ligament; metal particles; a platelet-rich plasma (PRO); a bone marrow aspirate concentrate (BMAC); a bone morphogenetic protein (BMP); and a combination thereof.

4. The system of claim 1, wherein the deformable structure comprises three or more expandable wings.

5. The system of claim 1, wherein the deformable structure comprises an outer tubular body and an inner tubular body that at least partially overlap, and wherein each of the tubular bodies comprises a plurality of deformable wings and wherein the outer tubular body comprises a plurality of longitudinal spaces configured to receive the wings of the inner tubular body when the deformable structure is in the expanded configuration, wherein the inner tubular body comprises inner wings and the outer tubular body comprises outer wings arranged alternately with the inner wings at the expanded configuration.

6. The system of claim 1, wherein the deformable structure comprises at least one stopper that prevents the wings from being overly deformed, wherein the stopper comprises opposing ribs configured to contact one another to limit contraction of the deformable structure.

7. The system of claim 1, wherein the deformable structure is made from a material selected from the group consisting of a shape memory material, a titanium, a cobalt chrome, a stainless steel, an alloy, a metal, a bio-absorbable material, a polymer, and a combination thereof.

8. The system of claim 1, wherein the bone anchoring device further comprises at least one suture wire coupler at its distal tip, the suture wire coupler is configured to couple at least one suture wire to the bone anchoring device.

9. The system of claim 8, wherein the suture wire coupler is disposed externally at said distal tip and said at least one suture wire extends from the distal tip along the exterior of the bone anchoring device, and/or wherein said suture wire coupler is disposed internally within said tip and said suture wire extending from the distal tip along the interior of the bone anchoring device.

10. The system of claim 1, further comprising at least one suture wire, wherein the at least one suture wire is slidable along the bone anchoring device, or fixedly attached thereto.

11. The system of claim 1, wherein the bone anchor body has an outer screw-like structure, or wherein the bone anchor body has a smooth outer surface.

12. The system of claim 1, wherein the target skeletal position is selected from a bone, a void space of a missing or a defected bone or a portion thereof, and a space between bone portions.

13. The system of claim 1, wherein the deformable structure is configured to deform following a pulling force applied on the distal tip to thereby deform the deformable structure to push out the wings so that the wings outwardly protrude from the bone anchoring device, wherein the deformable structure is configured to deform when a pulling force is applied by a delivery tool of the bone anchoring device.

14. The system of claim 1, further comprising a delivery tool for inserting and/or deploying within the bone a bone anchoring device, comprising:
a delivery shaft configured to couple to the bone anchoring device;
a delivery handle connected to a proximal end of the delivery shaft;
a delivery slider disposed within the delivery shaft and configured to move axially within the delivery shaft; and
a rotation mechanism on the delivery handle operatively coupled to the delivery slider, the rotation mechanism configured to move the delivery slider to expand the deformable structure of the bone anchoring device.

15. The system of claim 14, further comprising a rotating member coupling the rotation mechanism to the delivery slider, wherein the rotating member is configured to convert rotational motion of the rotation mechanism into linear motion of the delivery slider.

16. The system of claim 14, wherein the delivery tool further comprises a deployment indicator configured to provide visual feedback on the position of the delivery slider.

17. The system of claim 14, wherein the delivery shaft comprises a shaft thread configured to engage with a corresponding first thread on a bone anchoring device, wherein the slider comprises a slider thread configured to engage with a corresponding second thread on a bone anchoring device, configured for simultaneous disengagement of both threads from the bone anchoring device, wherein the shaft thread and the slider thread have similar thread pitches allowing for simultaneous disengagement of both threads from the bone anchoring device.

18. The system of claim 17, wherein the delivery tool is configured to detach from the bone anchoring device through a swiveling motion, the swiveling motion allows simultaneous disengagement of the shaft thread of the shaft from the first thread of the bone anchoring device and the slider thread on the slider from a second thread of the bone anchoring device.

19. The system of claim 14, further comprising a mechanism for retrieving the bone anchoring device out of the target skeletal position wherein said mechanism is configured to attach to the bone anchoring device in the bone, and to flatten the deformable structure in the target skeletal position wherein said mechanism comprises:
a shaft configured for insertion into a lumen of the body, the distal tip of the shaft configured to couple to the bone anchoring device; and
a handle for maneuvering said mechanism to remove the bone anchoring device from within the target skeletal position.

20. A method of performing a bone surgery comprising:
providing a bone anchoring system according to claim 1;
drilling into a bone to form a lumen that is dimensioned to receive at least a portion of the bone anchoring device;
inserting at least the distal tip of the bone anchoring device into the lumen of the bone; and
deforming the deformable structure of the bone anchoring device to thereby laterally expand the deformable wings of the deformable structure against the walls of the lumen of the bone.
